SEQUENCE LISTING (14 sequences)

SEQ ID NO: 1 - protein, 266 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS

SEQ ID NO: 2 - protein, 119 aa, Homo sapiens
QTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKL

SEQ ID NO: 3 - protein, 89 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQPDDVYYC

SEQ ID NO: 4 - protein, 23 aa, Homo sapiens
EASNCFAIRHFENKFAVETLICS

SEQ ID NO: 5 - protein, 95 aa, Homo sapiens
FADSDPANIVHDFNKKLTAYLDLNLDKCYVIPLNTSIVMPPRNLLELLINIKAGTYLPQSYLIHEHMVITDRIENIDHLGFFIYRLCHDKETYKL

SEQ ID NO: 6 - protein, 95 aa, Pan troglodytes; identical to SEQ ID NO: 5

SEQ ID NO: 7 - protein, 95 aa, Bos taurus
FADSDPANIVHDFNKKLTAYLDLNLDKCYVIPLNTSIVMPPKNLLELLINIKAGTYLPQSYLIHEHMVITDRIENIDHLGFYIYRLCHDKETYKL

SEQ ID NO: 8 - protein, 95 aa, Sus scrofa
FADSDPANIVHDFNKKLTAYLDLNLDKCYVIPLNTSIVMPPRNLLELLINIKAGTYLPQSYLIHEHMVITDRIENIDHLGFYIYRLCHDKETYKL

SEQ ID NO: 9 - protein, 95 aa, Mus musculus
FADSDPANIVHDFNKKLTAYLDLNLDKCYVIPLNTSIVMPPKNLLELLINIKAGTYLPQSYLIHEHMVITDRIENVDNLGFFIYRLCHDKETYKL

SEQ ID NO: 10 - protein, 95 aa, Rattus norvegicus
FADSDPANIVHDFNKKLTAYLDLNLDKCYVIPLNTSIVMPPRNLLELLINIKAGTYLPQSYLIHEHMVITDRIENVDHLGFFIYRLCHDKETYKL

SEQ ID NO: 11 - protein, 127 aa, Homo sapiens
  DOMAIN 3..121: note = Rh Bri2 BRICHOS domain
  DOMAIN 122..127: note = AU1 tag
GSQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLDTYRYI

SEQ ID NO: 12 - protein, 363 aa, Homo sapiens
  DOMAIN 3..121: note = Rh Bri2 BRICHOS domain
  DOMAIN 129..363: note = mCherry
GSQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLGGGGGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 13 - protein, 119 aa, synthetic construct
QTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYELCHDKETYKL

SEQ ID NO: 14 - protein, 95 aa, synthetic construct
FADSDPANIVHDFNKKLTAYLDLNLDKCYVIPLNTSIVMPPRNLLELLINIKAGTYLPQSYLIHEHMVITDRIENIDHLGFFIYELCHDKETYKL

(19) United States
(12) Patent Application Publication
(10) Pub. No.: US 2026/0347447 A1
(43) Pub. Date: Nov. 5, 2026

(54) THE BRI2 BRICHOS DOMAIN FOR DELIVERY OF PROTEINS INTO CNS NEURONS

(71) Applicant: ALPHABETA AB, Djursholm (SE)

(72) Inventor: Jan JOHANSSON, Stockholm (SE)

(21) Appl. No.: 19/120,509

(22) PCT Filed: Oct. 11, 2023
(86) PCT No.: PCT/EP2023/078258

§ 371 Date: Apr. 11, 2025

(30) Foreign Application Priority Data
Oct. 12, 2022 (US) 18046138

(51) Int. Cl. C07K 16/18; A61K 38/00; A61P 25/28; C07K 14/705

(52) U.S. Cl. CPC C07K 16/18; A61K 38/00; A61P 25/28; C07K 14/705; C07K 2319/03

(57) ABSTRACT

An isolated protein comprises (i) a first protein moiety selected from a group of proteins comprising an amino acid sequence having at least 70% identity to residues 113-231 of Bri2 from human (SEQ ID NO: 2); and proteins comprising an amino acid sequence having at least 70% identity to a BRICHOS domain of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); and (ii) a second protein or polypeptide moiety selected from a group of protein drugs, polypeptide drugs, protein tags, fluorescent proteins, antibodies, enzymes and/or neurotrophic factors. The isolated protein comprises an amino acid sequence having neither at least 70% identity to residues 1-89 of Bri2 from human (SEQ ID NO: 3) nor at least 70% identity to human ABri23 (SEQ ID NO: 4).

### FIELD OF THE INVENTION

The present invention pertains to the field of medicine. More specifically, this invention relates to use of substances and agents for improved medical treatment of Alzheimer's disease, in mammals, such as man. The invention furthermore relates to substances and agents for transporting a protein across the blood-brain barrier in a mammal, including man. The invention also relates to delivery of proteins into CNS neurons and treatment of neurological diseases.

### BACKGROUND TO THE INVENTION

An increasing number of neurodegenerative conditions are linked to protein misfolding and aggregation, such as Alzheimer's disease and familial British or Danish dementia. These diseases are characterized by protein deposits in the brain parenchyma and cerebral arteries, and occur in inherited and sporadic forms. Even though these diseases have different clinical symptoms, they share some common pathological features, such as neuronal loss, protein aggregates, and presence of tau tangles. From a biochemical point of view, the proteins involved have a tendency to form β-sheet structures and are prone to aggregate into amyloid fibrils. Alzheimer's disease and familial British or Danish dementia display several similar neuropathological hallmarks. Amyloid plaques, neurofibrillary tangles, Congophilic amyloid angiopathy and neurodegeneration are observed. Alzheimer's disease is one of the most common causes of dementia in man. It is a chronic and fatal disease associated with neural cell degeneration in the brain of the affected individual, characterized by the presence of amyloid plaques consisting of extracellular deposits of amyloid β-peptide (Aβ-peptide). The neural cell atrophy caused by Aβ aggregation results in deficiency of acetylcholine and other signalling substances. It is known that Aβ-peptide, having 40-42 amino acid residues, is produced by processing of the amyloid precursor protein (APP, 695-770 amino acid residues), which is a type I membrane protein normally expressed by the neurons of the central nervous system, but the reasons for this processing are incompletely understood. The released Aβ peptide contains a part of the transmembrane region of APP (Aβ residues 29-40/42) and includes a discordant helix, i.e. a helix composed of amino acids with a high propensity to form β-strands. Aβ is prone to misfold and aggregate when removed from its stabilising membrane environment.

Bri2 (SEQ ID NO: 1, also referred to as integral membrane protein 2B, ITM2B), is a 266-residue type II membrane protein (FIG. 1) with ubiquitous expression, whose function and folded structure are unknown. Bri2 is proteolytically cleaved at three locations; cleavage by furin in the C-terminal region generates a 23-residue peptide (ABri23), processing of the ectodomain by ADAM10 results in release of the BRICHOS domain from the membrane-bound N-terminal part, and intramembrane cleavage by SPPL2a/2b liberates the intracellular domain. Familial British and Danish dementia are caused by mutations in the Bri2 gene that result in a loss of a stop codon, which in turn results in two different 11-residue extensions of the C-terminal part, and, after furin cleavage, generation of 34-residue peptides (ABri and ADan, respectively) instead of the normally released ABri23. The longer peptides are prone to aggregation into amyloid fibrils and deposition in brain tissue or cerebral vessels, with concomitant neuronal loss and dementia.

Recent studies have shown that Bri2 and Aβ co-localize in amyloid plaques in brain parenchyma and vessels, suggesting that the proteins interact at some stage during misfolding and aggregation. Using transfected cell lines, Bri2 has been found to interact with APP, and to modulate APP processing by increasing β-secretase generated fragments. Generation of a fusion protein containing Bri2 and Aβ40 indicates that the Bri protein can affect Aβ aggregation properties, and using a transgenic mouse model, ABri23 has been proposed to interact with Aβ42 and prevent its aggregation (Kim et al. J. Neurosci. 28:6030-6036 (2008); WO 2009/009396). It has also been suggested that Aβ production can be reduced or prevented by a protein containing the first 102 amino acid residues of Bri2 (WO 2006/138355).

The BRICHOS domain is a naturally occurring chaperone with anti-amyloid properties found in 10 different human proprotein families, one (proSP-C) of which is associated with amyloid lung disease, and one (Bri2/ITM2b) that is as earlier described associated with the amyloid related dementias familial British or Danish dementia. Recombinant human (rh) BRICHOS domains from proSP-C and Bri2 delay Aβ40 and Aβ42 fibril formation and reduce the neurotoxicity associated with Aβ42 fibril formation in vitro and in vivo (WO 2011/62655).

The blood-brain barrier (BBB) functions to maintain a delicate homeostasis required for proper neuronal function. The BBB also functions as a barrier towards substances and agents targeting the brain, larger molecules are unable to spontaneously cross the BBB. The BBB nevertheless presents an efficient pathway for the transportation of compositions such as agents, drugs and biologic drugs, such as proteins, into the central nervous system (CNS). Only small and lipophilic molecules have been shown to be able to pass passively across the BBB. Recent studies have shown that isolated recombinant Bri2 BRICHOS proteins with an attached AU1 tag for immunodetection (Bri2 BRICHOS-AU1) can be detected in the brain parenchyma after peripheral (intravenous) administration to wild type mice, while recombinant proSP-C BRICHOS proteins only pass into the cerebrospinal fluid (CSF). This suggests that an unknown mechanism for transport of Bri2 BRICHOS over the BBB exists (Mikitsh et al. Perspect Medicin Chem. 6:11-24 (2014); Sanchez-Covarrubias et al. Curr Pharm Des. 20:1422-49 (2014); Tambaro et al. J Biol Chem. 294:2606-2615 (2019)).

Various methods have been developed for delivery of compositions over the BBB. Focused ultrasound combined with intravenous lipid microbubbles (MBs) results in a localized and reversible opening of the BBB, which allows transport of macromolecules including proteins into the brain parenchyma surrounding the area that is targeted by ultrasound (Mikitsh et al. Perspect Medicin Chem. 6:11-24 (2014); Brasnjevic et al. Prog Neurobiol. 87:212-51 (2009); Konofagou et al. Theranostics. 2:1223-37 (2012); Sierra et al. J Cereb Blood Flow Metab. 37:1236-1250 (2017)). These methods require specialized ultrasound equipment. It has also been considered that the ultrasound treatment may under certain conditions give rise to vascular damage.

Biverstål et al., Scientific Reports, 10:21765 (2020) discloses functionalization of amyloid fibrils using fusion proteins containing a Bri2 BRICHOS domain, which is reported as useful to synthesize amyloid decorated with different protein functionalities.

Current therapeutic approaches for treatment of Alzheimer's disease are mainly directed to treating the symptoms and include cholinergic replacement therapy, e.g. inhibition of acetylcholinesterase, small inhibitors that interact with soluble Aβ oligomers, and so-called β-sheet breakers that prevent elongation of already formed β-sheet structures. Furthermore, it is of interest to be able to lower the doses of the therapeutic drugs in order to reduce side-effects associated with ultrasound treatments in combination with other treatments.

### SUMMARY OF THE INVENTION

It is an object of the invention to provide a new treatment option for the treatment of Alzheimer's disease in a mammal, including man.

It is also an object of the invention to provide a robust and facilitated method and means for efficient delivery of proteins, such as therapeutic agents, over the blood brain barrier and into the CNS. In particular, it is an object of the invention to achieve delivery of proteins, such as therapeutic agents, into CNS neurons.

One object of the invention is to provide a simple and efficient method and means for efficient delivery of Bri2 BRICHOS and variants thereof over the blood-brain barrier in the treatment of Alzheimer's disease.

It is another object of the invention to decrease the tendency of proteins that are prone to fibrillate to aggregate into amyloid fibrils, or even prevent proteins that are prone to fibrillate from aggregating into amyloid fibrils.

It is yet another object to decrease formation of amyloid plaques consisting of extracellular deposits in the brain of a mammal of proteins that are prone to fibrillate.

It is an object of the invention to impinge on the distribution of isolated recombinant Bri2 BRICHOS and variants thereof so that the therapeutically effective amount of isolated recombinant Bri2 BRICHOS reaching the brain is increased to efficiently combat Aβ42 neurotoxicity.

It is another object of the invention to provide a protein and a method for improved treatment, in vivo diagnostics and prognostics, and/or imaging of Alzheimer's disease and other neurological diseases in a mammal, including man.

It is a further object of the invention to provide novel proteins and a method for treatment of Parkinson's disease in a mammal, including man.

The present invention is generally based on the insight that the isolated recombinant protein Bri2 BRICHOS and variants thereof can be efficiently delivered over the blood-brain barrier when administered in combination with lipid microbubbles and/or nanodroplets, without any step of ultrasound treatment of any tissue of a mammal. It is surprising that co-administration with lipid microbubbles and/or nanodroplets achieves an improved uptake of Bri2 BRICHOS and variants thereof in absence of ultrasound treatment, compared to both (a) in the absence of lipid microbubbles and/or nanodroplets, and (b) in the presence of ultrasound treatment. The method is not comprising any step of treatment of any tissue of the mammal with optical, audio or ultrasonic waves which make the microbubbles and/or nanodroplets cavitate in the tissue.

Furthermore, the present invention is also based on the insight that microbubbles and/or nanodroplets alone, in the absence of ultrasound treatment, may be used to enhance the delivery of proteins comprising Bri2 BRICHOS and variants thereof as a first protein moiety coupled to another (non-Bri2) second protein or polypeptide moiety over the blood-brain barrier and thereby e.g. facilitate treatment and/or diagnostics of Alzheimer's disease and other neurological diseases involving the second protein or polypeptide moiety.

One aspect of the present invention is based on the insight that proteins comprising the isolated recombinant protein Bri2 BRICHOS and variants thereof, including Bri2 BRICHOS R221E, can be efficiently delivered over the blood-brain barrier into CNS neurons. The method is not requiring any step of administering lipid microbubbles and/or nanodroplets. The method is not requiring any step of treatment of any tissue of the mammal with optical, audio or ultrasonic waves. Bri2 BRICHOS R221E is described in WO 2021/140140 A1, which is incorporated herein in its entirety by reference.

For these and other objects that will be evident from the following description and the appended claims, the present invention provides according to a first aspect an isolated recombinant protein comprising Bri2 BRICHOS and variants thereof for use in a method of treatment of Alzheimer's disease. According to a second aspect, there is provided a method of treating Alzheimer's Disease comprising administrating an isolated recombinant protein comprising Bri2 BRICHOS and variants thereof and lipid microbubbles and/or nanodroplets without any ultrasound treatment.

According to a third aspect, there is provided a protein comprising a first protein moiety which is Bri2 BRICHOS and variants thereof and a second protein or polypeptide moiety, and a combination thereof with lipid microbubbles and/or nanodroplets. According to a fourth aspect, this combination is useful in a method for transporting the protein across the blood-brain barrier in a mammal without any ultrasound treatment.

The third aspect of a protein comprising a first protein moiety which is Bri2 BRICHOS and variants thereof and a second protein or polypeptide moiety is advantageously useful for increasing transport of the second protein or polypeptide moiety over the blood-brain barrier. The first (Bri2 BRICHOS) moiety aids in the transport, and this is achieved without lipid microbubbles and/or nanodroplets. One particularly useful variant of the first protein moiety is then Bri2 BRICHOS R221E since it is demonstrated herein to improve the transport compared to wildtype Bri2 BRICHOS. The transport can be further improved with lipid microbubbles and/or nanodroplets. If lipid microbubbles and/or nanodroplets are included, the method does not require any step of treatment of any tissue of the mammal with optical, audio or ultrasonic waves which make the microbubbles and/or nanodroplets cavitate in the tissue.

According to a further aspect, there is provided a treatment of Parkinson's Disease protein comprising administration of Bri2 BRICHOS and variants thereof. There is further provided a protein comprising a first protein moiety which is Bri2 BRICHOS and variants thereof and a second protein or polypeptide moiety, wherein the second protein or polypeptide moiety is itself effective for treatment of Parkinson's Disease. This combination is also useful for treatment of Parkinson's Disease.

### BRIEF DESCRIPTION OF THE DRAWINGS

FIG. 1 shows a schematic outline of Bri2 (SEQ ID NO: 1) and processing sites.

FIG. 2 shows an alignment of some mammalian Bri2 BRICHOS amino acid sequences (SEQ ID NOS: 5-10).

FIG. 3 shows the study design in the Examples.

FIG. 4 shows that Bri2 BRICHOS, but not proSP-C BRICHOS, is detected in non-FUS-targeted brain hemisphere after i.v. injection together with lipid microbubbles and/or nanodroplets.

FIG. 5 shows that Bri2 BRICHOS is detected in both hemispheres after i.v. injection together with lipid microbubbles and/or nanodroplets.

FIG. 6 shows intracellular immunostaining for Bri2 BRICHOS-AU1 in the cortex and hippocampus after i.v. injection together with lipid microbubbles and/or nanodroplets.

FIG. 7 shows western blot detection of Bri2 BRICHOS-AU1 in both brain hemispheres after systemic injection together with lipid microbubbles and/or nanodroplets.

FIG. 8 shows that Rh Bri2 BRICHOS-mCherry is found in the striatum after systemic injection in combination with lipid microbubbles and/or nanodroplets.

FIG. 9 illustrates detection of Bri2 BRICHOS in AppNL-G-F and AppNL-F mouse brains after repeated injections of Bri2 BRICHOS R221E.

FIG. 10 shows hCMEC/D3 monolayer permeability of different proteins and macromolecules.

FIG. 11 shows staining with anti-NT antibody of brain cortex of mice injected with NT-Bri2 BRICHOS (A) or PBS (B).

FIG. 12 illustrates that Rh Bri2 BRICHOS mediates uptake into mouse primary neurons.

FIG. 13 shows TEM images of α-syn fibrils after fibrillization at 70 μM without (a) or with 100% (b) BRICHOS molar equivalent. (c) Fibril diameter of α-syn fibril incubated without or with BRICHOS.

FIG. 14 illustrates that BRICHOS monomers prevents a neurotoxic effect of sonicated α-syn fibrils.

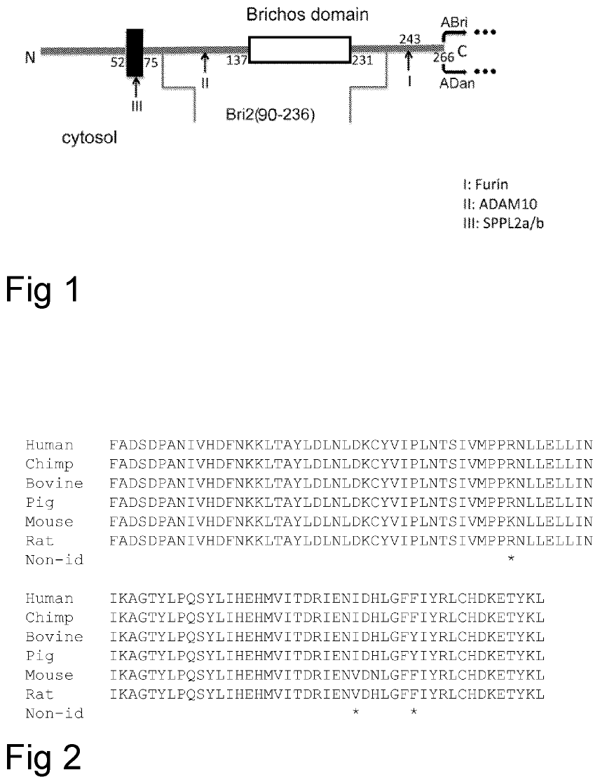

List of appended sequences

| | | |
|---|---|---|
| SEQ ID | human Bri2 | MVKVTFNSAL AQKEAKKDEP KSGEEALIIP |
| NO: 1 | | PDAVAVDCKD PDDVVPVGQR RAWCWCMCFG |
| | | LAFMLAGVIL GGAYLYKYFA LQPDDVYYCG |
| | | IKYIKDDVIL NEPSADAPAA LYQTIEENIK |
| | | IFEEEEVEFI SVPVPEFADS DPANIVHDFN |
| | | KKLTAYLDLN LDKCYVIPLN TSIVMPPRNL |
| | | LELLINIKAG TYLPQSYLIH EHMVITDRIE |
| | | NIDHLGFFIY RLCHDKETYK LQRRETIKGI |
| | | QKREASNCFA IRHFENKFAV ETLICS |
| SEQ ID | human Bri2 | QTIEENIKIF EEEEVEFISV PVPEFADSDP |
| NO: 2 | (113-231) | ANIVHDENKK LTAYLDLNLD KCYVIPLNTS |
| | | IVMPPRNLLE LLINIKAGTY LPQSYLIHEH |
| | | MVITDRIENI DHLGFFIYRL CHDKETYKL |
| SEQ ID | human Bri2(1-89) | MVKVTFNSAL AQKEAKKDEP KSGEEALIIP |
| NO: 3 | | PDAVAVDCKD PDDVVPVGQR RAWCWCMCFG |
| | | LAFMLAGVIL GGAYLYKYFA LQPDDVYYC |
| SEQ ID | human ABri23 | EASNCFAIRH FENKFAVETL ICS |
| NO: 4 | [Bri2(244-266)] | |
| SEQ ID | human Bri2_{BRICHOS} | FADSDPANIV HDFNKKLTAY LDLNLDKCYV |
| NO: 5 | [Bri2(137-231)] | IPLNTSIVMP PRNLLELLIN IKAGTYLPQS |
| | | YLIHEHMVIT DRIENIDHLG FFIYRLCHDK |
| | | ETYKL |
| SEQ ID | chimpanzee | FADSDPANIV HDFNKKLTAY Y LDLNLDKCYV |
| NO: 6 | Bri2_{BRICHOS} | IPLNTSIVMP PRNLLELLIN IKAGTYLPQS |
| | | YLIHEHMVIT DRIENIDHLG FFIYRLCHDK |
| | | ETYKL |
| SEQ ID | bovine Bri2_{BRICHOS} | FADSDPANIV HDFNKKLTAY LDLNLDKCYV |
| NO: 7 | | IPLNTSIVMP PKNLLELLIN IKAGTYLPQS |
| | | YLIHEHMVITD RIENIDHLG FYIYRLCHDK |
| | | ETYKL |
| SEQ ID | pig Bri2_{BRICHOS} | FADSDPANIV HDFNKKLTAY LDLNLDKCYV |
| NO: 8 | | IPLNTSIVMP PRNLLELLIN IKAGTYLPQS |
| | | YLIHEHMVIT DRIENIDHLG FYIYRLCHDK |
| | | ETYKL |
| SEQ ID | mouse Bri2_{BRICHOS} | FADSDPANIV HDFNKKLTAY LDLNLDKCYV |
| NO: 9 | | IPLNTSIVMP PKNLLELLIN IKAGTYLPQS |
| | | YLIHEHMVIT DRIENVDNLG FFIYRLCHDK |
| | | ETYKL |
| SEQ ID | rat Bri2_{BRICHOS} | FADSDPANIV HDFNKKLTAY LDLNLDKCYV |
| NO: 10 | | IPLNTSIVMP PRNLLELLIN IKAGTYLPQS |
| | | YLIHEHMVIT DRIENVDHLG FFIYRLCHDK |
| | | ETYKL |
| SEQ ID | rh Bri2 BRICHOS- | GSQTIEENIK IFEEEEVEFI SVPVPEFADS |
| NO: 11 | AU1 | DPANIVHDEN KKLTAYLDLN LDKCYVIPLN |
| | | TSIVMPPRNL LELLINIKAG TYLPQSYLIH |
| | | EHMVITDRIE NIDHLGFFIY RLCHDKETYK |
| | | LDTYRYI |
| SEQ ID | rh Bri2 BRICHOS- | GSQTIEENIK IFEEEEVEFI SVPVPEFADS |
| NO: 12 | mCherry | DPANIVHDEN KKLTAYLDLN LDKCYVIPLN |
| | | TSIVMPPRNL LELLINIKAG TYLPQSYLIH |
| | | EHMVITDRIE NIDHLGFFIY RLCHDKETYK |
| | | LGGGGGGSVS KGEEDNMAII KEFMRFKVHM |
| | | EGSVNGHEFE IEGEGEGRPY EGTQTAKLKV |
| | | TKGGPLPFAW DILSPQFMYG SKAYVKHPAD |
| | | IPDYLKLSFP EGFKWERVMN FEDGGVVTVT |
| | | QDSSLQDGEF IYKVKLRGTN FPSDGPVMQK |
| | | KTMGWEASSE RMYPEDGALK GEIKQRLKLK |
| | | DGGHYDAEVK TTYKAKKPVQ LPGAYNVNIK |
| | | LDITSHNEDY TIVEQYERAE GRHSTGGMDE |
| | | LYK |
| SEQ ID | rh Bri2 BRICHOS | QTIEENIKIF EEEEVEFISV PVPEFADSDP |
| NO: 13 | R221E | ANIVHDENKK LTAYLDLNLD KCYVIPLNTS |
| | | IVMPPRNLLE LLINIKAGTY LPQSYLIHEH |
| | | MVITDRIENI DHLGFFIYEL CHDKETYKL |
| SEQ ID | human Bri2_{BRICHOS} | FADSDPANIV HDFNKKLTAY LDLNLDKCYV |
| NO: 14 | [Bri2(137-231)] | IPLNTSIVMP PRNLLELLIN IKAGTYLPQS |
| | R221E | YLIHEHMVIT DRIENIDHLG FFIYELCHDK |
| | | ETYKL |

### DETAILED DESCRIPTION OF THE INVENTION

Bri2 (SEQ ID NO: 1), also referred to as integral membrane protein 2B (ITM2B), contains an evolutionary conserved BRICHOS domain spanning residues 137-231 (SEQ ID NO: 5). BRICHOS domains are found in more than different protein families that are functionally unrelated and expressed in different tissues. The name BRICHOS refers to identification of the domain in Bri, chondromodulin-1 related to chondrosarcoma and in lung surfactant protein C precursor (proSP-C) involved in respiratory disease.

10 Proteins comprising the BRICHOS domain of a mammalian Bri2 (ITM2B) and structurally similar proteins have the capacity to decrease amyloid fibril formation and aggregation of Aβ-peptide and ABri/ADan peptides.

The blood-brain barrier (BBB) functions to maintain a delicate homeostasis required for proper neuronal function, the BBB also functions as a barrier towards substances and agents targeting the brain, larger molecules are unable to spontaneously cross the BBB. The BBB nevertheless presents an efficient pathway for the transportation of pharmaceutical compositions as proteins into the central nervous system (CNS). Only small and lipophilic molecules have been shown to be able to pass passively across the BBB.

The present invention is generally based on the insight that an isolated Bri2 BRICHOS protein as such, or coupled to a second protein or polypeptide moiety, can be efficiently delivered into the brain in combination with lipid microbubbles and/or nanodroplets and would thus, be useful for treating Alzheimer's disease.

Furthermore, an isolated Bri2 BRICHOS protein coupled to a second (cargo) protein or polypeptide moiety, can be efficiently delivered into the brain, even without lipid microbubbles and nanodroplets.

The lipid microbubbles and/or nanodroplets and the isolated protein may be administered in combination. The term “combination” and/or “combination”, as used herein refers to that the isolated proteins and the lipid microbubbles and/or nanodroplets may be administered individually in any order independently of each other. The term may also refer to that the recombinant proteins and the lipid microbubbles and/or nanodroplets may be administered individually and at the same time. Furthermore, the term may refer to that the isolated proteins and lipid microbubbles and/or nanodroplets may be administered in the same pharmaceutical composition.

The isolated protein according to the invention can be incorporated into pharmaceutical compositions. Such compositions typically include the isolated protein according to the invention and a suitable pharmaceutically acceptable carrier. As used herein, a “suitable pharmaceutical carrier” includes solvents, dispersion media, coatings, isotonic and absorption delaying agents, and the like, that are compatible with pharmaceutical administration. Supplementary active compounds can also be incorporated into the compositions.

In prior art methods, ultrasound is an important local stimulus for triggering drug release at the target tissue and may consist of pressure waves at frequencies of 20 KHz or greater. Like optical and audio waves, ultrasonic waves are focused, reflected, and refracted through a medium.

Microbubbles and/or nanodroplets in combination with ultrasound treatment are typically used in medical diagnostics and non-invasive delivering of pharmaceutical compositions and genes to different tissues. Focused ultrasound combined with intravenously administered microbubbles and/or nanodroplets is a technology that has been shown in multiple in vivo models to efficiently deliver small- and large-molecules over the BBB and to a specifically targeted brain region in a minimally invasive way. This technique involves administration of lipid-based microbubbles and/or nanodroplets together with the pharmaceutical composition to be delivered. The ultrasonic waves make the microbubbles and/or nanodroplets cavitate within the capillaries in the brain. At these low pressures, microbubbles and/or nanodroplets exhibit stable cavitation which induces an increase in the opening of the BBB. This makes the tight junctions between the endothelial cells loosen transiently, which results in transient and local opening of the BBB. As a result, macromolecules present in the circulation are delivered into the brain parenchyma (Galan-Acosta et al. Mol Cell Neurosci, 103498 (2020)).

Here, we design a facilitated method for efficient delivery of a composition over a tissue such as the blood brain barrier and into neurons in the CNS. This method combines the administration of an isolated protein with the administration of lipid microbubbles and/or nanodroplets. Importantly, the present method does not involve any step of ultrasound treatment. This is advantageous as it does not require any ultrasound equipment. Furthermore, side effects associated with ultrasound treatment, e.g. vascular damage, can be avoided. It is surprising that co-administration with lipid microbubbles and/or nanodroplets achieves an improved uptake of Bri2 BRICHOS and variants thereof in absence of ultrasound treatment, compared to both (a) in the absence of lipid microbubbles and/or nanodroplets, and (b) in the presence of ultrasound treatment.

The method is not comprising any step of treatment of any tissue of the mammal with optical, audio or ultrasonic waves which make the microbubbles and/or nanodroplets cavitate in the tissue.

For these and other objects that will be evident from the following description, the present invention provides according to a first aspect an isolated recombinant protein selected from the group of proteins consisting of an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4); for use in a method of treatment of Alzheimer's disease in a mammal, including man, in need thereof comprising the steps of;
administrating to said mammal a plurality of lipid microbubbles and/or nanodroplets
administrating to said mammal said isolated recombinant protein;
wherein said isolated recombinant protein is not comprised within said microbubbles and/or nanodroplets; and
wherein the method is not comprising any step of ultrasound treatment of any tissue of the mammal.

It is experimentally shown herein that the lipid microbubbles and/or nanodroplets alone, i.e. in the absence of ultrasound treatment, are able to mediate uniform transfer of Bri2 BRICHOS over the BBB, and that Bri2 BRICHOS is delivered into the brain parenchyma and is efficiently taken up by neurons in the cortex as well as the hippocampus when the administration of Bri2 BRICHOS and lipid microbubbles and/or nanodroplets is combined.

It has surprisingly been found that Bri2 BRICHOS proteins are delivered to wildtype mice brain when administered in combination with microbubbles and/or nanodroplets. This is particularly surprising in view of the various methods that have previously been developed, wherein focused ultrasound combined with intravenous lipid microbubbles and/or nanodroplets are used to deliver drugs and other compositions over the BBB by allowing transport of macromolecules including proteins into the brain parenchyma surrounding the area that is targeted with ultrasound.

The present invention is based on the herein disclosed, surprising insights that the microbubbles and/or nanodroplets on their own, without being combined with any step of ultrasound treatment of any tissue of the mammal have the capacity to mediate increased transfer of the isolated recombinant protein over the BBB. Microbubbles and/or nanodroplets increase the passage of Bri2 BRICHOS over the BBB and uptake in neurons.

This aspect of the invention is advantageous in that transient and local opening of the BBB can be obtained without the use of any additional treatment steps such as ultrasound treatments and additional equipment associated with such additional treatments. Thus, a facilitated method for efficient delivery of a composition over a tissue such as the blood brain barrier and into neurons in the CNS is provided. Another advantage is, as shown herein, that the uptake of the isolated recombinant proteins into the brain parenchyma is highly increased when administered in combination with lipid microbubbles and/or nanodroplets in comparison with administrating the isolated recombinant proteins alone as previously described in WO 2011/162655.

The term “% similarity”, as used throughout the specification and the appended claims, is calculated as described for “% identity”, with the exception that the hydrophobic residues Ala, Val, Phe, Pro, Leu, Ile, Trp, Met and Cys are similar; the basic residues Lys, Arg and His are similar; the acidic residues Glu and Asp are similar; and the hydrophilic, uncharged residues Gln, Asn, Ser, Thr and Tyr are similar. The remaining natural amino acid Gly is not similar to any other amino acid in this context.

Throughout this description, alternative embodiments fulfil, instead of the specified percentage of identity, the corresponding percentage of similarity. Other alternative embodiments fulfil the specified percentage of identity as well as another, higher percentage of similarity, selected from the group of preferred percentages of identity for each sequence. For example, the isolated recombinant protein sequence may be 70% similar to another protein sequence; or it may be 70% identical to another sequence; or it may be 70% identical and furthermore 90% similar to another sequence.

For avoidance of doubt, the amino acid sequence having at least the given identity to residues 113-231 of Bri2 from human or any one of the BRICHOS domains of Bri2 consists of more than or equal to 70, such as more than or equal to 80, such as more than or equal to 90 amino acid residues. A preferable size range is 70-100 amino acid residues, such as 80-100 amino acid residues, e.g. 90-100 amino acid residues.

It is noted that the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10) is highly conserved, see alignment in FIG. 2. Without desiring to be bound to any specific theory, it is contemplated that the BRICHOS domain harbours the desired activity with respect to the Aβ and ABri/ADan peptides. It is preferred that the isolated recombinant protein according to the invention is selected from the group consisting of proteins comprising an amino acid sequence having at least 80%, preferably at least 90%, such as at least 95%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10). In a preferred embodiment, the isolated recombinant protein according to the invention contains all amino acid residues that are conserved in the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10), i.e. all amino acid residues of SEQ ID NO: 5 except for residues 42, 76 and 82 (corresponding to residues 178, 212 and 218 in full length Bri2, SEQ ID NO: 1). In specific embodiments, the isolated recombinant protein according to the invention is selected from the group consisting of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10), i.e. it contains one of these BRICHOS domains, preferably the human BRICHOS domain (SEQ ID NO: 5).

In a preferred embodiment, the amino acid residue corresponding to position 221 in SEQ ID NO: 1 is selected from the group consisting of Glu and Asp. In a specific embodiment, the amino acid residue corresponding to position 221 in SEQ ID NO: 1 is Glu. We mutated rh Bri2 BRICHOS so that the monomer is stabilised relative to larger oligomers and rh Bri2 BRICHOS R221E (SEQ ID NO: 13) selectively reduces Aβ42 oligomer generation and alleviates Aβ42-induced neurotoxicity in hippocampal slice preparations. Rh Bri2 BRICHOS R221E passes the BBB in mice and shows a trend towards higher passage than the wildtype protein. This result is in line with the previous observation that monomeric wildtype rh Bri2 BRICHOS passes the BBB more efficiently than larger oligomers.

In contrast to previous teachings, the isolated recombinant protein according to the invention is not comprising an amino acid sequence having at least 70% identity to residues 1-89 of Bri2 from human (SEQ ID NO: 3). In certain embodiments, the isolated recombinant protein according to the invention is not comprising an amino acid sequence having at least 50% identity to residues 1-89 of Bri2 from human (SEQ ID NO: 3). This implies that the isolated recombinant protein according to the invention contains a core amino acid sequence which displays a high similarity or identity to residues 113-231 of Bri2 from human (SEQ ID NO: 2) and/or a mammalian BRICHOS domain of Bri2 from (SEQ ID NOS: 5-10) and optionally one or more other amino acid sequences, which other amino acid sequences may not display a high similarity or identity to residues 1-89 of Bri2 from human (SEQ ID NO: 3).

For avoidance of doubt, amino acid sequences that are shorter than 10 amino acid residues are not considered relevant in the context of being excluded from the isolated recombinant protein according to the invention. Thus, the isolated recombinant protein according to the invention is not comprising an amino acid sequence that consists of more than or equal to 10 amino acid residues having at least the given identity to residues 1-89 of Bri2 from human (SEQ ID NO: 3).

Furthermore, the isolated recombinant protein according to the invention is not comprising an amino acid sequence having at least 70% identity to residues 244-266 of Bri2 from human, i.e. human ABri23 (SEQ ID NO: 4). In certain embodiments, the isolated recombinant protein according to the invention is not comprising an amino acid sequence having at least 50% identity to residues human ABri23 (SEQ ID NO: 4). As set out above, this implies that the isolated recombinant protein according to the invention contains a core amino acid sequence which displays a high similarity or identity to residues 113-231 of Bri2 from human (SEQ ID NO: 2) and/or a mammalian BRICHOS domain of Bri2 from (SEQ ID NOS: 5-10) and optionally one or more other amino acid sequences, which other amino acid sequences may not display a high similarity or identity to human ABri23 (SEQ ID NO: 4).

For avoidance of doubt, amino acid sequences that are shorter than 10 amino acid residues are not considered relevant in the context of being excluded from the isolated recombinant protein according to the invention. Thus, the isolated recombinant protein according to the invention is not comprising an amino acid sequence that consists of more than or equal to 10 amino acid residues having at least the given identity to human ABri23 (SEQ ID NO: 4).

In preferred embodiments, the isolated recombinant protein for use according to the invention is selected from the group consisting of proteins comprising an amino acid sequence having at least 70% identity to residues 113-231 of Bri2 from human (SEQ ID NO: 2); and proteins comprising an amino acid sequence having at least 70% identity to the BRICHOS domain of Bri2 from human (SEQ ID NO: 5).

In a preferred embodiment an isolated recombinant protein selected 5 from the group of proteins consisting of an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4); for use in a method of treatment of Alzheimer's disease in a mammal, including man, in need thereof consisting of the steps of;
administrating to said mammal a plurality of lipid microbubbles and/or nanodroplets
administrating to said mammal said isolated recombinant protein;
wherein said isolated recombinant protein is not comprised within said microbubbles and/or nanodroplets.

In specific embodiments a combination of an isolated recombinant protein and lipid microbubbles and/or nanodroplets for use in a method of treatment of Alzheimer's disease in a mammal, including man, in need thereof is comprising the steps of;
administrating to said mammal a plurality of lipid microbubbles and/or nanodroplets
administrating to said mammal said isolated recombinant protein;
wherein said isolated recombinant protein is not comprised within said microbubbles and/or nanodroplets; and
wherein the method is not comprising any step of ultrasound treatment of any tissue of the mammal.

In a specific embodiment a combination of an isolated recombinant protein and lipid microbubbles and/or nanodroplets for use in a method of treatment of Alzheimer's disease in a mammal, including man, in need thereof is consisting of the steps of;
administrating to said mammal a plurality of lipid microbubbles and/or nanodroplets
administrating to said mammal said isolated recombinant protein;
wherein said isolated recombinant protein is not comprised within said microbubbles and/or nanodroplets.

The pharmaceutical composition comprising the isolated recombinant protein may be useful as a medicament, specifically in treatment of conditions such as Alzheimer's disease in a mammal, including man.

A pharmaceutical composition is formulated to be compatible with its intended route of administration. Examples of routes of administration include parenteral (e.g. intravenous, intraarterial), intraperitoneal, intramuscular, intradermal and intranasal.

Administration of the microbubbles and/or nanodroplets and the isolated recombinant protein may include injections.

Sterile injectable solutions can be prepared by incorporating the isolated recombinant protein according to the invention in the required amount in an appropriate solvent with one or a combination of ingredients enumerated above, as required, followed by filtered sterilization. Generally, dispersions may be prepared by incorporating the isolated recombinant protein according the invention into a sterile vehicle which contains a dispersion medium and other ingredients required. In the case of sterile powders for the preparation of sterile injectable solutions, the preferred methods of preparation are vacuum drying and freeze-drying, which yield a powder of the isolated recombinant protein according the invention plus any additional desired ingredient from a previously sterile-filtered solution thereof.

Generally, sterile injectable solutions of lipid microbubbles and/or nanodroplets may be prepared, as set out e.g. in Feshitan, J. A. et al., J. Colloid Interface Sci. 329 (2), 316-324 (2009). Briefly, the gas perfluorobutane (PFB) may be used to form the microbubbles and/or nanodroplets. PFB may act as a gas core, that can be introduced in order to activate the lipid microbubbles and/or nanodroplets, and which are later isolated. The microbubbles and/or nanodroplets may be coated with 1,2-Distearoyl-sn-glycero-3-phosphocholine (DSPC) and Polyoxyethylene-40 stearate (PEG40S) DSPC and PEG40S. The obtained dried lipid film may be hydrated with filtered PBS and mixed to a final lipid suspension.

The lipid mixture may be sonicated in order to disperse the lipid aggregates into small, unilamellar liposomes. PFB gas may be introduced by flowing it over the surface of the lipid suspension. Subsequently, higher power sonication may be applied to the suspension at the gas-liquid interface to generate microbubbles and/or nanodroplets. Following isolation, the microbubbles and/or nanodroplets according to the invention may be incorporated into a sterile vehicle e.g. the microbubble and/or nanodroplet suspension may be collected into 30-mL syringes, washing and size fractionating may be achieved by centrifugation in order to collect all microbubbles and/or nanodroplets from the suspension into a cake resting against the syringe plunger. The remaining suspension (infranatant), which may contain residual lipids and vesicles that did not form part of the microbubble shells, may be recycled to produce the next batch of microbubbles and/or nanodroplets. All resulting cakes may be combined and re-suspended in PBS to improve total yield.

In certain embodiments said isolated recombinant protein and said lipid microbubbles and/or nanodroplets are administered intravenously.

When an isolated protein according to the invention is to be administered to an animal (e.g. a human) to treat Alzheimer's disease, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific isolated recombinant protein employed, the age, body weight, general health, gender, and diet of the subject, the time of administration, the route of administration, the rate of excretion, any drug combination, and the degree of expression or activity to be modulated.

Data obtained from cell culture assays and animal studies may be used in formulating a range of dosage for use in humans.

The dosage may vary within this range depending upon the dosage form employed and the route of administration utilized. For any compound used in the method of the invention, the therapeutically effective dose can be estimated initially from cell culture assays in which, e.g. the rate of fibril formation or the rate of cell death is observed. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an isolated recombinant protein according to the invention (i.e., an effective dosage) ranges from about 1 to 50 mg/kg body weight. In one embodiment, a therapeutically effective amount of at least 1 mg/kg, such as at least 5 mg/kg, more preferably such as at least 10 mg/kg of said isolated recombinant protein is administered.

In another embodiment, a therapeutically effective amount of less than 50 mg/kg, such as less than 30 mg/kg, more preferably less than 20 mg/kg of the isolated recombinant protein is administered.

The isolated recombinant protein can be administered over an extended period of time to the subject, e.g., over the subject's lifetime. A dosage of 1 mg/kg to 50 mg/kg body weight is usually appropriate. By administrating the isolated recombinant protein in combination with the lipid microbubbles and/or nanodroplets according to the invention administration of significantly lower doses of the isolated recombinant protein than previously described in WO 2011/162655 is enabled. The administration of lipid microbubbles and/or nanodroplets results in the localized and reversible opening of the BBB, which allows for efficient transport and increased uptake of the isolated recombinant protein into the brain parenchyma. This aspect of the invention is also advantageous since the risk of side effects associated with administration of a therapeutic composition for both prophylactic and therapeutic methods of treating a subject who has or is at risk of (or susceptible to) Alzheimer's disease is minimized.

In some cases, the lipid microbubbles and/or nanodroplets and the isolated recombinant protein can be administered once per week for between about 1 to 10 weeks, preferably between 2 to 8 weeks, more preferably between about 3 to 7 weeks, and even more preferably for about 4,5, or 6 weeks. The compound can also be administered chronically. The skilled person will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount lipid microbubbles and/or nanodroplets and the isolated recombinant protein can include a single treatment or, preferably, can include a series of treatments.

In further embodiments, the isolated protein is selected from the group consisting of proteins comprising an amino acid sequence having at least 70%, preferably at least 75%, 80%, 85%, 90%, 95%, or 99% identity to residues 113-231 of Bri2 from human (SEQ ID NO: 2); and proteins comprising an amino acid sequence having at least 70%, preferably at least 75%, 80%, 85%, 90%, 95%, or 99% identity to the BRICHOS domain of Bri2 from human (SEQ ID NO: 5).

In preferred embodiments, the isolated protein is selected from the group consisting of residues 113-231 of Bri2 from human (SEQ ID NO: 2); and the BRICHOS domain of Bri2 from human (SEQ ID NO: 5).

According to a related aspect, the present invention provides an isolated selected from the group of proteins comprising an amino acid sequence having at least 70%, preferably at least 75%, 80%, 85%, 90%, 95%, or 99% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In another embodiment the isolated protein is selected from the group consisting of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In specific embodiments, the isolated protein is selected from the group consisting of any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In certain embodiments, the isolated protein consists of less than or equal to 200 amino acid residues, such as less than or equal to 150 amino or even 100 amino acid residues. In certain embodiments, the isolated protein consists of more than or equal to 90 amino acid residues, such as more than or equal to 100 amino acid residues.

A preferable size range of the isolated protein is 80-200 amino acid residues, such as 90-150 amino acid residues, e.g. 90-100 amino acid residues.

Microbubbles and/or nanodroplets are small gas-filled microspheres. They may consist of gas surrounded by a by a lipid, lipopolymer, or polymer shell. They may also be similar in size to red blood cells and may range from 0.5-10 μm.

The gas-filled microbubbles and/or nanodroplets, oscillate and vibrate when a sonic energy field is applied. Microbubbles and/or nanodroplets having a hydrophilic outer layer to interact with the bloodstream and a hydrophobic inner layer to house the gas molecules are the most thermodynamically stable. Air, sulfur hexafluoride, and perfluorocarbon gases may serve as the composition of the microbubble interior. For increased stability and persistence in the bloodstream, gases with high molecular weight as well as low solubility in the blood are attractive candidates for microbubble gas cores. Microbubbles and/or nanodroplets may be used for drug delivery, and they may not only serve as drug vehicles but also as a means to permeate otherwise impenetrable barriers, specifically the blood brain barrier.

In further embodiments of the invention the microbubbles and/or said nanodroplets are lipid coated.

As disclosed herein, the microbubbles and/or nanodroplets may comprise 1,2-distearyol-sn-glycero-3-phosphocoline (DSPC), 1,2-distearyol-sn-glycero-3-phosphoethanolamine-N-(metoxy (polyethyleneglycol) 2000) and a gas core of perfluorobutane. The microbubbles and/or nanodroplets may e.g. comprise sulphur hexafluoride, polyethylene glycol (PEG, Macrogol), distearylphosphatidylcholine (DSPC), sodium 1,2-dipalmitoyl-sn-glycero-3-phosphatidylglycerol and palmitic acid.

In a preferred embodiment of the invention the individual microbubbles and/or nanodroplets according to the invention have a diameter in the range of 1-8 μm, such as 2-6 μm, such as 4-5 μm.

The skilled person will appreciate that the embodiments discussed above in relation to the first aspect of the present disclosure, are equally relevant and applicable to the second, third and further aspects disclosed herein. This particularly applies to embodiments relating to the isolated recombinant protein and the lipid microbubbles and/or nanodroplets, the inventive combination underlying the efficient and uniform transfer of the recombinant isolated protein over the BBB and into the brain parenchyma, as well as the efficient uptake by the neurons in the cortex and the hippocampus, and to embodiments relating to the mode and route of administration. For the sake of brevity these will not be repeated here or will only be briefly mentioned.

In a second aspect of the invention, there is provided a method of treating Alzheimer's disease in a mammal, including man, in need thereof comprising the steps of;
administrating to said mammal a plurality of lipid microbubbles and/or nanodroplets
administrating to said mammal an isolated recombinant protein selected from the group of proteins consisting of an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4);
wherein said isolated recombinant protein is not comprised within said microbubbles and/or nanodroplets; and wherein the method is not comprising any step of ultrasound treatment of any tissue of the mammal.

In embodiments of the second aspect, a method of treating Alzheimer's disease in a mammal, including man, in need thereof is provided, consisting of the steps of;
administrating to said mammal a plurality of lipid microbubbles and/or nanodroplets
administrating to said mammal an isolated recombinant protein selected from the group of proteins consisting of an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4);
wherein said isolated recombinant protein is not comprised within said microbubbles and/or nanodroplets.

As a further aspect of the invention, there is provided an isolated recombinant protein consisting of residues 113-231 of Bri2 from human (SEQ ID NO: 2.

The present invention provides for both prophylactic and therapeutic methods of treating a subject at risk of (or susceptible to) Alzheimer's disease. As used herein, the term “treatment” is defined as the application or administration of an isolated recombinant protein and lipid microbubbles and/or nanodroplets according to the invention to a patient, or application or administration of an isolated recombinant protein and lipid microbubbles and/or nanodroplets according to the invention to an isolated tissue or cell line from a patient, who has Alzheimer's disease, a symptom of disease or a predisposition toward a disease, with the purpose to cure, heal, alleviate, relieve, alter, remedy, ameliorate, improve or affect the disease, the symptoms of disease or the predisposition toward disease. In specific embodiments, the treatment is selected from the group consisting of preventive, palliative and curative treatment.

In one aspect, the invention provides a method for preventing a disease or condition (i.e., decreasing the risk of contracting, or decreasing the rate at which symptoms appear that are associated with a disease or condition) associated with fibril formation caused by Aβ peptide and/or ABri/ADan peptide by administering to the subject an isolated recombinant protein and lipid microbubbles and/or nanodroplets according to the invention that reduces aggregation of the polypeptide. Subjects at risk for Alzheimer's disease can be identified by, for example, any or a combination of appropriate diagnostic or prognostic assays known in the art. Administration of a prophylactic agent can occur prior to the manifestation of symptoms characteristic of the disease, such that the disease is prevented or, alternatively, delayed in its progression.

The isolated recombinant protein and the lipid microbubbles and/or nanodroplets according to the invention can be administered to a patient at therapeutically effective doses to prevent, treat or ameliorate disorders involving fibril formation associated with Alzheimer's disease. A therapeutically effective dose refers to that amount of the compound sufficient to result in amelioration of symptoms of the disorders. Toxicity and therapeutic efficacy of such compounds can be determined by standard pharmaceutical procedures as described above.

According to a different aspect of the invention, the Bri2 BRICHOS protein and variants thereof are useful in the delivery of protein or polypeptides such as therapeutic agents, antibodies and protein tags by providing distinct advantage of improving for example therapeutic potential of drugs and drug targeting, in vivo diagnostics and prognostics, and in vivo imaging. A protein may thus comprise at least one further protein or polypeptide moiety, wherein the protein moiety may be a biological polymer, an oligomer and an oligopeptide such as peptides and polypeptides.

As a third aspect of the invention, there is provided an isolated protein comprising
(i) a first protein moiety selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); and
(ii) a second protein or polypeptide moiety, preferably containing at least 50 amino acid residues;
wherein said isolated protein is not comprising an amino acid sequence having at least 70% identity to residues 1-89 of Bri2 from human (SEQ ID NO: 3); and
wherein said isolated protein is not comprising an amino acid sequence having at least 70% identity to human ABri23 (SEQ ID NO: 4).

Proteins comprising the first protein moieties are unique in their in vivo therapeutic application by providing tissue-specific targeting and/or release of drugs for use in treatment and therapies, in vivo diagnostics and prognostics, and in vivo imaging when used in combination with microbubbles and/or nanodroplets.

The Bri2 BRICHOS domain provides the capacity to transport the second protein or polypeptide moiety across the blood-brain barrier in a mammal without any ultrasound treatment. An isolated protein comprising a Bri2 BRICHOS domain and second protein or polypeptide moiety such as protein drugs, polypeptide drugs, protein tags, fluorescent proteins, antibodies, enzymes and/or neurotrophic factors is advantageous since it facilitates and enhances the treatment of Alzheimer's Disease and other neurological diseases.

In an embodiment, the first protein moiety of the isolated protein is selected from the group of proteins comprising an amino acid sequence having at least 70%, preferably at least 80%, 85%, 90%, 95% or 99%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In another embodiment, the first protein moiety is selected from the group of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In one embodiment of the invention the first protein moiety of the isolated protein is selected from the group of proteins comprising an amino acid sequence having at least 70%, preferably at least 80%, 85%, 90%, 95% or 99%, identity to any one of residues 113-231 of Bri2 from human (SEQ ID NO: 2) and the BRICHOS domain of Bri2 from human (SEQ ID NO: 5).

In a further embodiment the first protein moiety is selected from the group consisting of residues 113-231 of Bri2 from human (SEQ ID NO: 2); and the BRICHOS domain of Bri2 from human (SEQ ID NO: 5).

In one embodiment, the amino acid residue in the first protein moiety corresponding to position 221 in SEQ ID NO: 1 is selected from the group consisting of Glu and Asp, preferably Glu. We mutated rh Bri2 BRICHOS so that the monomer is stabilised relative to larger oligomers and rh Bri2 BRICHOS R221E (SEQ ID NO: 13-14) selectively reduces Aβ42 oligomer generation and alleviates Aβ42-induced neurotoxicity in hippocampal slice preparations. Rh Bri2 BRICHOS R221E passes the BBB in mice and shows a trend towards higher passage than the wildtype protein. This result is in line with the previous observation that monomeric wildtype rh Bri2 BRICHOS passes the BBB more efficiently than larger oligomers. The improved propensity of the R221D/E variant for passage is particularly advantageous when efficiently delivery into the brain is desired, without co-administration of lipid microbubbles and nanodroplets.

In one embodiment, the first protein moiety is consisting of less than or equal to 200 amino acid residues, such as less than or equal to 150 amino acid residues. In one embodiment, the first protein moiety is consisting of more than or equal to 90 amino acid residues.

Generally, delivery of large therapeutics molecules into the brain to treat central nervous system (CNS) diseases is a major drug development challenge. The BBB serves to restrict movement of substances from the circulating blood to the CNS. Thus, only approximately 0.1% of circulating antibodies cross the intact BBB, severely limiting the therapeutic utility of antibody therapeutics for CNS disorders. Furthermore, the BBB excludes from the brain 100% of large-molecule neurotherapeutics and more than 98% of all small-molecule drugs.

In one embodiment, the isolated protein does not contain a cleavage site between the first protein moiety and the Bri2-BRICHOS sequence and the second protein or polypeptide moiety. In another embodiment, the isolated protein contains a cleavage site between the first protein moiety and the Bri2-BRICHOS sequence and the second protein or polypeptide moiety, e.g. the cleavage site in Bri2 which in the native protein is naturally cleaved by proprotein convertases to release Abri peptide.

An object of the invention is to provide an isolated protein wherein the second protein or polypeptide moiety of the isolated protein contains from 50 to 2000 amino acid residues, such as from 50 to 1000 amino acid residues, such as from 50 to 500 amino acid residues, such as from 50 to 100 amino acid residues. In another object of the invention the size of the second protein or polypeptide moiety of the isolated protein is 5-200 kDa, such as 5-100 kDa, such as 5-50 kDa, such as 5-10 kDa.

The inventors have surprisingly found that an isolated protein comprising Bri2 BRICHOS and a second (cargo) protein or polypeptide moiety can cross the BBB despite its large size.

In some embodiments of the invention, the first protein moiety of the isolated protein is linked directly or indirectly to the amino-terminal or the carboxy-terminal end of the second protein or polypeptide moiety.

In another embodiment of the invention the second protein or polypeptide moiety of the isolated protein constitutes the amino-terminal and/or the carboxy-terminal end of the isolated protein.

As previously mentioned, the BBB often hinders the brain delivery of large therapeutic molecules such as protein drugs, polypeptide drugs, protein tags, fluorescent proteins, antibodies, enzymes and/or neurotrophic factors.

Protein and polypeptide drugs can be used to replace a protein that is abnormal or deficient in a particular disease. They can also augment the body's supply of a beneficial protein to help reduce the impact of diseases treatments. Examples of common and widely used protein drugs are insulin, Interferon alpha and Interleukin-2.

Protein tags are peptide sequences grafted onto a protein, e.g. genetically grafted onto a recombinant protein. Examples of protein tags include solubilization tags, epitope tags and fluorescence tags.

Solubilization tags are used, especially for recombinant proteins expressed in chaperone-deficient species such as E. coli, to assist in the proper folding in proteins and keep them from precipitating.

Epitope tags are short peptide sequences which are chosen because high-affinity antibodies can be reliably produced in many different species.

Fluorescence tags and proteins are used to give visual readout on a protein. They may be used to tag components in a cell, a tissue or an organ so they can be studied using fluorescence spectroscopy, fluorescence microscopy and other imaging techniques. GFP and its variants are the most commonly used fluorescence tags. mCherry on the other hand is a member of the mFruits family of monomeric red fluorescent proteins (mRFPs) and belongs to the group of fluorescent protein chromophores used as instruments to visualize genes and analyze their functions in experiments.

The second protein moiety can also increase the stability of the isolated protein and the first protein moiety which itself may be useful as a therapeutical agent, e.g. increase half-life in the body. Thereby, the frequency of treatments may be decreased. One possibility is that the second protein moiety is an Fc moiety, i.e. the fragment crystallizable region (Fc region) from the tail region of an antibody that interacts with cell surface receptors called Fc receptors and some proteins of the complement system.

In another embodiment, the second protein or polypeptide moiety of the isolated protein is selected from the group consisting of protein drugs, polypeptide drugs, protein tags, fluorescent proteins, antibodies, enzymes and/or neurotrophic factors.

Antibodies, also known as an immunoglobulin (lg) are large, Y-shaped proteins used by the immune system to identify and neutralize foreign objects such as pathogenic bacteria and viruses. Using their binding mechanism antibodies are widely used in therapies, wherein they are employed to treat diseases such as rheumatoid arthritis, multiple sclerosis, psoriasis, and many forms of cancers. Monoclonal antibodies that have been studied and/or are used for treatment of Alzheimer's disease are aducanumab, gantenerumab, 3D6 (bapineuzumab) and m266 (solanezumab).

In a preferred embodiment, the second protein or polypeptide moiety of the isolated protein is an antibody, such as a monoclonal antibody. Preferred monoclonal antibodies are selected from (i) aducanumab, gantenerumab, 3D6 (bapineuzumab), m266 (solanezumab), donanemab and lecanemab or (ii) aducanumab, gantenerumab, 3D6 (bapineuzumab), and m266 (solanezumab). A preferred monoclonal antibody is donanemab. Another preferred monoclonal antibody is lecanemab.

Neurotrophic factors (NTFs) are a family of biomolecules, nearly all of which are peptides or small proteins that support the growth, survival, and differentiation of both developing and mature neurons. Neurotrophic factors also promote the initial growth and development of neurons in the central nervous system and peripheral nervous system, and they are capable of regrowing damaged neurons in test tubes and animal models. Some neurotrophic factors are also released by the target tissue in order to guide the growth of developing axons. In studies, neurotrophic factors are normally used in conjunction with other techniques, neurotrophic factors may be immobilized to a scaffold structure. In neural drug delivery systems, they are loosely immobilized such that they can be selectively released at specified times and in specified amounts.

In an embodiment, the second protein or polypeptide moiety of the isolated protein is a neurotrophin selected from the group consisting of brain-derived neurotrophic factor (BDNF), nerve growth factor (NGF), neurotrophin-3, neurotrophin-4, ciliary neurotrophic factor (CNTF), glial cell line-derived neurotrophic factor (GDNF), ephrins, epidermal growth factor (EGF), transforming growth factor (TGF), insulin-like growth factor (IGF), vascular endothelial growth factor (VEGF), fibroblast growth factor (FGF), platelet-derived growth factor (PDGF), and/or interleukins.

Enzymes are proteins that act as biological catalysts and accelerate chemical reactions. Enzymes are required for many chemical interconversions that support life and speed up all the biochemical processes in the body. These characteristics distinguish them from other types of drugs. Due to these characteristics, enzymes are widely used medically either alone or adjunctly with other therapies, with the purpose of safe treatment of various diseases. Examples of therapeutic enzymes used for treatment and different therapies of various disorders are α-L-iduronidase, Iduronate sulfatase, N-acetylgalactosamine 6-sulfatase, N-acetylgalactosamine 4-sulfatase, α-galactosidase, α-glucosidase, β-glucocerebrosidase and/or Lysosomal acid lipase. Further examples of therapeutic enzymes which can constitute the second protein include enzymes known to be genetically mutated, and therefore functionally deficient in lysosomal storage diseases (LSDs).

In another embodiment, the second protein or polypeptide moiety of the isolated protein is selected from the group consisting of α-L-iduronidase, Iduronate sulfatase, N-acetylgalactosamine 6-sulfatase, N-acetylgalactosamine 4-sulfatase, α-galactosidase, α-glucosidase, β-glucocerebrosidase and/or Lysosomal acid lipase.

Fusion proteins are proteins created through the joining of two or more genes that originally coded for separate proteins. Translation of these fusion genes results in a single protein with functional properties derived from each of the original proteins. Recombinant fusion proteins are created artificially by recombinant DNA technology for use in biological research or therapeutics.

In some embodiments of the invention the isolated protein is a recombinant fusion protein.

Chemical linking is the process of chemically joining two or more protein molecules such as chemically linking a first protein moiety to a second protein or polypeptide moiety. Chemical linking enables attachment of a moiety such as a protein, polypeptide, protein drug, tag and fluorescent molecules to another protein molecule in order to transport to and target cells and tissues, providing target specific therapy and treatments, and/or improved in vivo diagnostics and prognostics, imaging and aid in detection of a molecule(s).

In another embodiment of the invention the first protein moiety of the isolated protein is chemically linked to said second protein or polypeptide moiety.

As a further aspect of the invention, there is provided a combination of an isolated protein according as disclosed above and a plurality of lipid microbubbles and/or nanodroplets, wherein the isolated protein is not comprised within said microbubbles and/or said nanodroplets.

In one embodiment, a kit comprising an isolated protein as disclosed above and a plurality of lipid microbubbles and/or nanodroplets is provided, wherein the isolated protein is not comprised within said microbubbles and/or said nanodroplets.

Brain-penetrating molecules, recombinant proteins and/or biologics are generally not successful unless the pharmaceutical crosses the BBB. Thus, the BBB drug delivery is the limiting factor in the future development of new therapeutics for the brain.

The inventors have designed a facilitated method for efficient delivery of a composition over a tissue such as the BBB and into neurons in the CNS.

For these and other objects, the present invention provides according to a further aspect of the invention a method for transporting an isolated protein as disclosed herein across the blood-brain barrier in a mammal, including man, in need thereof consisting of the steps of;
administrating to said mammal a plurality of lipid microbubbles and/or nanodroplets; and
administrating to said mammal said isolated protein.

In a specific embodiment the method does not comprise any step of ultrasound treatment of any tissue of the mammal. The present invention provides according to a yet further aspect an isolated protein as disclosed herein for use in a method of treatment involving transporting said isolated protein across the blood-brain barrier in a mammal, including man, in need thereof comprising the steps of:
administrating to said mammal a plurality of lipid microbubbles and/or nanodroplets;
administrating to said mammal said isolated protein;
wherein said isolated protein is not comprised within said microbubbles and/or said nanodroplets.

In a certain embodiment, the method is not comprising any step of ultrasound treatment of any tissue of the mammal.

The method is not comprising any step of treatment of any tissue of the mammal with optical, audio or ultrasonic waves which make the microbubbles and/or nanodroplets cavitate in the tissue.

According to another aspect, a method for transporting an isolated protein as disclosed herein across the blood-brain barrier in a mammal, including man, in need thereof is comprising, or consisting of, the step of; administrating to said mammal said isolated protein.

In a specific embodiment, there is provided an isolated protein as disclosed herein for use in a method of treatment involving transporting said protein across the blood-brain barrier in a mammal, including man, in need thereof consisting of the steps of:
administrating to said mammal a plurality of lipid microbubbles and/or nanodroplets; and
administrating to said mammal said isolated protein;
wherein said isolated protein is not comprised within said microbubbles and/or said nanodroplets.

It has surprisingly been found that both rh Bri2 BRICHOS fused to a short peptide tag or to a globular protein were detected in the brain parenchyma 2 hours after administration together with lipid microbubbles and/or nanodroplets. Analyses of the brain parenchyma by immunohistochemistry and western blot clearly show similar distribution of delivered protein in both hemispheres, with strong staining in the cortex, hippocampus and the choroid plexus after administration of rh Bri2 BRICHOS-AU1 with microbubbles and/or nanodroplets. The internalization by cells of rh Bri2 BRICHOS-AU1 delivered together with microbubbles and/or nanodroplets was seen, which is consistent with results obtained from FUS-mediated delivery of rh Bri2 BRICHOS-AU1 (Galan-Acosta et al., Mol Cell Neurosci. 2020:103498). Bri2 is expressed not only in the CNS but also in peripheral tissues, and the existence of a transport system that allows the crosstalk between both sites could be envisioned.

The transport of drugs into cerebrospinal fluid (CSF) cannot be extrapolated to BBB passage, and in line with that notion it can be noted that another BRICHOS protein, rh proSP-C BRICHOS, was detected in CSF after intravenous injection in mice, but was not found in brain homogenates. This indicates that passage of the blood CSF barrier does not ensure transport over the BBB or into the brain parenchyma (Tambaro et al. J Biol Chem. 294: 2606-2615 (2019)). The permeability of the BBB differs between different brain regions, for example in areas close to the choroid plexus and in the circumventricular areas the permeability is higher than in the rest of the brain.

Moreover, quantitative analysis of total rh Bri2 BRICHOS domain that reached the brain parenchyma by sandwich ELISA shows considerable amounts of rh Bri2 BRICHOS-AU1 in both hemispheres. According to this analysis, 1% of the totally injected rh Bri2 BRICHOS domain administered with lipid microbubbles and/or nanodroplets is detected in the brain 2 hours after injection, which is between 2-10 fold more than the passage observed when rh Bri2 BRICHOS-AU1 was administered without microbubbles and/or nanodroplets (Tambaro et al., J Biol Chem. 2019; 294 (8): 2606-15) or with microbubbles and/or nanodroplets plus FUS (Galan-Acosta et al., Mol Cell Neurosci. 2020:103498). In the case of the rh Bri2 BRICHOS-mCherry fusion protein, the magnitude of the increased BBB passage mediated by microbubbles and/or nanodroplets was not quantified, but the results indicate an effect as the fusion protein could only be detected in the brain when it was administered together with microbubbles and/or nanodroplets.

Taken together, the current data demonstrate that microbubbles and/or nanodroplets significantly increase the passage of rh Bri2 BRICHOS over the BBB, also when it is fused to a large, 30 kDa globular protein. Incorporation or association of drugs with lipid nanoparticles can increase bioavailability, but in the present invention the rh Bri2 BRICHOS proteins and microbubbles and/or nanodroplets are administered independently, making it unlikely that rh Bri2 BRICHOS passes the BBB together with microbubbles and/or nanodroplets. Without desiring to be bound by any particular theory, the inventors suspect that increased passage of rh Bri2 BRICHOS, or rh Bri2 BRICHOS-mCherry, over the BBB in the presence of microbubbles and/or nanodroplets is mediated by a prolonged half-life of rh Bri2 BRICHOS in the circulation. Other means to increase the half-life of rh Bri2 BRICHOS in the circulation will most likely increase its passage over the BBB and rh Bri2 BRICHOS fused to other proteins, including for example antibodies, enzymes or neurothrophic factors may increase their passage into the brain parenchyma, as functional mCherry now was shown to be able to be transported into the brain of wildtype mice.

The findings of the present invention show that the extent of rh Bri2 BRICHOS passage through the BBB into the brain parenchyma in the presence of microbubbles and/or nanodroplets is higher (1% of injected dose) than what have been observed for peripherally administered antibodies (about 0.1% of injected dose) (Bard et al. Nat Med. 2000; 6 (8): 916-919 and Zucheroet al. 2016; 89 (1): 70-82). Rh Bri2 BRICHOS BBB passage is likewise apparently more efficient than affibodies designed to improve CNS uptake, which were only detected in low amounts in the cerebrospinal fluid after injection (Meister et al. Int J Mol Sci. 2020; 21 (8)).

As set out above, proteins comprising the isolated recombinant protein Bri2 BRICHOS and variants thereof, including Bri2 BRICHOS R221E, can be efficiently delivered over the blood-brain barrier into CNS neurons. The method is not requiring any step of administering lipid microbubbles and/or nanodroplets, The method is not requiring any step of treatment of any tissue of the mammal with optical, audio or ultrasonic waves. Thus, the isolated Bri2 BRICHOS protein coupled to a second protein or polypeptide moiety, can be efficiently delivered into the brain, even without lipid microbubbles and 5 nanodroplets.

We show that cargo proteins fused to rh Bri2 BRICHOS pass a human BBB model made of cerebral microvascular endothelial cells and astrocytes as efficiently as proteins known to be transported over the BBB. In contrast, 4 kDa dextran or the fusion partners alone do not pass, and rh Bri2 BRICHOS does not cause unspecific leakage or cell toxicity. Intravenously injected fusion protein with Bri2 BRICHOS passes the mouse BBB and colocalizes with early endosomes in cerebral and cerebellar neurons. Our results show that fusions with rh Bri2 BRICHOS can providep multimodal biologic drugs for CNS disorders.

As demonstrated herein, an isolated protein comprising a first protein moiety which is a Bri2 BRICHOS protein or a variant thereof and at least one further protein or polypeptide moiety is useful in a method for transporting the isolated protein across the blood-brain barrier in a mammal, including man, in need thereof comprising, or consisting of, the step of:

administrating to said mammal said isolated protein.

Furthermore, the isolated protein comprising a first protein moiety which is a Bri2 BRICHOS protein or a variant thereof and at least one further protein or polypeptide moiety is useful in a method of treating a medical condition in a mammal, including man, in need thereof comprising, or consisting of, the step of:
administrating to said mammal said isolated protein.

Accordingly, the isolated protein is useful as a medicament. The isolated protein is useful in a method of treating a medical condition in a mammal, including man, in need thereof comprising, or consisting of, the step of:
administrating to said mammal said isolated protein.

In these methods, the isolated protein comprising a first protein moiety which is a Bri2 BRICHOS protein or a variant thereof and at least one further protein or polypeptide moiety can be efficiently delivered into the brain, even without lipid microbubbles and nanodroplets.

It is also contemplated that the isolated proteins according to the invention can be administrated by gene therapy, such as by using expression vectors, plasmids or viruses to transfect cells in the neural system, preferably brain, such that the isolated protein is expressed by these cells in the central neural system. This is useful for the treatment of Alzheimer's disease. This may also be useful for treatment of other neurological diseases.

An isolated Bri2 BRICHOS protein as such can also be efficiently delivered into the brain where it is useful for treating Parkinson's disease.

Alpha-synuclein (α-syn) protein aggregation has been linked to several neurodegenerative disorders including Parkinson's disease (PD), dementia with Lewy bodies and multiple system atrophy, which are characterized by abnormal accumulation of misfolded α-syn into intracellular inclusions in neurons called Lewy bodies. The specific molecular mechanisms underlying the onset of the disease are still unknown but there is strong evidence that α-syn aggregation into amyloid fibrils plays a crucial role since known mutations in the gene coding for α-syn cause early onset or more aggressive forms of PD. Immense efforts have been undertaken to suppress amyloid formation in PD but no effective treatment has been developed so far. In particular, ways to inhibit toxic aggregation pathways and species are demanded, since the generation of intermediate, oligomeric α-syn assemblies have been suggested to cause α-syn associated neurotoxic effects.

Without wishing to be limited to any particular mechanism, it is demonstrated herein that BRICHOS efficiently delays α-synuclein self-assembly by binding to the fibril surface and thereby specifically suppresses, besides fibril-end elongation, secondary nucleation events and associated oligomer generation. Further, our findings give evidence that BRICHOS directly binds to oligomeric species and diminishes toxic effects associated to α-synuclein.

We show that the BRICHOS domain is able to prevent α-syn aggregation in a concentration dependent manner by affecting the secondary nucleation and elongation of WT α-syn. Interestingly, a similar inhibitory mechanism was reported for BRICHOS on Aβ42 aggregation (Chen et al., Nat Commun, 8 (1): 2081, 2017; Chen et al., Communications biology, 3 (1): 1-12, 2020). Previous studies have shown that BRICHOS binds along the fibril surface and Aβ42 fibrils (Biverstål et al., Sci Rep, 10 (1): 21765, 2020). One hypothetical mechanism would be that BRICHOS, while binding to secondary nucleation spots along the fibril surface, competes with α-syn monomers as shown with Aβ42.

We show for the first time the interaction between BRICHOS and α-syn oligomeric species. By binding to α-syn oligomers and fibrils, BRICHOS not only interferes with secondary nucleation but also bind to its products often correlated with toxicity in in vitro experiments. This binding ability could be crucial for reducing oligomer-induced toxicity.

While oligomers are the most toxic species, sonicated α-syn fibrils also show toxicity in different in vitro and in vivo models. Here, we show that BRICHOS prevents α-syn sonicated fibrils related neurotoxicity in mouse hippocampal slices. This could be explained by the fact that BRICHOS can bind to α-syn fibrils and supress the formation of oligomers through secondary nucleation process with endogenous α-syn. BRICHOS could also interfere with oligomeric species released from existing α-syn fibrils ends which display toxic behaviour in neuronal cells.

Based on relevant in vitro models, BRICHOS is useful as a treatment candidate against alpha-synucleopathies including Parkinson's Disease.

The Bri2 BRICHOS protein can also be coupled to a second protein or polypeptide which is effective for treatment of Parkinson's Disease, such as glucocerebrosidase, progranulin, prosaposin, cathepsin D and antibodies. Without wishing to be bound to any specific theory, it is believed that the combined effect of the Bri2 BRICHOS and the second protein/polypeptide will be highly useful in the treatment of Parkinson's Disease. It is also contemplated that the high efficiency of this agent will require less frequent administrations thereof.

Accordingly, there is provided a method of treating alpha-synucleopathies, including Parkinson's Disease, in a mammal, including man, in need thereof comprising, or consisting of, the step of:
administrating to said mammal an isolated protein as defined herein.

Furthermore, there is provided a method for delaying accumulation of α-synuclein in a mammal, including man, in need thereof comprising, or consisting of, the step of:
administrating to said mammal an isolated protein as defined herein.

The present invention will now be further illustrated by the following non-limiting examples.

### EXAMPLES

The Examples demonstrate that microbubbles can be used to enhance the delivery of Bri2 BRICHOS and fusion proteins thereof over the BBB and facilitate treatment of AD and other neurological diseases.

### Expression and Purification of Recombinant Proteins

Rh Bri2 BRICHOS domain, corresponding to residues 113-231 of the full length Bri2 protein, was linked to an AU1 tag or the mCherry protein at the C-terminal end. The BRICHOS containing proteins Bri2 BRICHOS-AU1 (SEQ ID NO: 11) and Bri2 BRICHOS-mCherry (SEQ ID NO: 12) were expressed in E. coli and purified. Before injection into mice, rh Bri2 BRICHOS-AU1 was dialyzed (6-8 kDa membrane, Spectrum lab) against filtered and autoclaved phosphate-buffer saline (PBS), pH 7.4. Endotoxins were eliminated by passing the proteins over a Pierce High-Capacity endotoxin removal column (Thermo scientific). The final protein preparations were filtered through a 0.22 μm Millex-GV filter (Millipore Ltd.) and they were stored at −20 °C and thawed a few minutes before injections.

As a control, a proSP-C BRICHOS domain (proSP-C residues 59-197) was produced as set out in Galan-Acosta et al., Mol Cell Neurosci. 2020:103498.

### Animals

Female and male C57BL/6J, age 4-6 months old, weight 24-30 g, were used. All animals were kept on 12-hour light-dark cycles and grouped in cages of 5 mice with food and water available ad libitum. All the experiments were approved and conducted in accordance with the ethical committee of Södra Stockholms Djurförsöksetiska Nämnd (dnr 03049), Linköpings Etiska Namnd (ID855) or under the guidelines of Columbia University Institutional Animal Care and Use Committee.

### Experimental Design

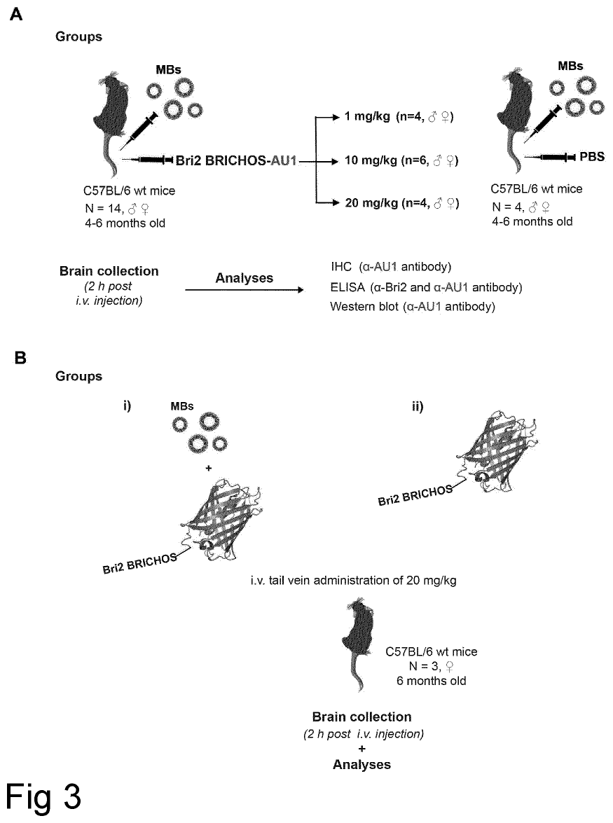

FIG. 3 shows the study design in the Examples. (A) Mice received microbubbles and 1, 10, or 20 mg/kg rh Bri2 BRICHOS-AU1 (SEQ ID NO: 11), or PBS for controls, double i.v. injections in the tail vein. (B) Mice received 20 mg/kg of (i) rh Bri2 BRICHOS-mCherry (SEQ ID NO: 12) and microbubbles, or (ii) rh Bri2 BRICHOS-mCherry only.

Two different types of commercially available microbubbles, either Definity® (Lantheus Medical Imaging, MA, USA) or SonoVue® (Bracco, Milan, Italy) were administered at a dose of 5 μl/g body weight. For one part of the study (FIG. 3A), a total of 14 mice were divided into four groups that received, respectively, i.v. injections of microbubbles and PBS (n=4), microbubbles and 1 (n=4), 10 (n=6) or 20 (n=4) mg/kg body weight of rh Bri2 BRICHOS-AU1 (SEQ ID NO: 11). In the second part of the study (FIG. 3B), a total of 3 mice were administered 20 mg/kg rh Bri2 BRICHOS-mCherry (SEQ ID NO: 12) (n=1) or microbubbles+20 mg/kg rh Bri2 BRICHOS-mCherry (n=2) intravenously. Two mice that received 10 mg/kg of rh Bri2 BRICHOS-AU1 were injected with Definity® microbubbles, the rest of the mice were administered with SonoVue® microbubbles.

All animals received two i.v. injections into the lateral tail vein by using a 29-gauge needle. Prior administration, microbubbles vials were activated with mechanical agitation for 20 seconds and the microbubbles solution was injected in the tail vein over 60 seconds, immediately followed by slow injections of rh Bri2 BRICHOS proteins or PBS controls. Before injections, mice were anesthetized using 2-4% isoflurane (carried with 2% oxygen) and put under a heat lamp in order to dilate the tail veins. Two hours after the injections, the mice were anesthetized and transcardially perfused with 120 ml PBS and one mouse, that was administered Definity® and 10 mg/kg of rh Bri2 BRICHOS-AU1, was perfused with PBS for 5 minutes followed by perfusion with 4% paraformaldehyde (PFA) for 7 minutes. The brains were extracted from skull and either snap-frozen in dry iced and stored at −80 °C or post-fixed in 4% PFA for 48 hours before being embedded in paraffin.

### Antibodies

For immunohistochemistry (IHC), polyclonal rabbit anti-AU1 (Abcam Cat #ab3401) primary antibodies were used at 1:200 dilution, and anti-rabbit secondary antibody conjugated with horseradish peroxidase (HRP) (GE Healthcare Cat #NA934) were diluted to 1:2000. For western blot, primary antibody was polyclonal rabbit anti-AU1 (Abcam Cat #ab3401) at 1:600 dilution and fluorescently labelled secondary anti-rabbit (Li-Cor, Cat #926-32213) antibody was used at 1:10,000 dilution. For sandwich ELISA, goat anti-Bri2 BRICHOS antibody was used for capture at 1:250 dilution, and polyclonal rabbit anti-AU1 (Abcam Cat #ab3401) antibodies were used for detection at 1:2000 dilution.

A rabbit anti-proSP-C antibody was used as previously reported in Galan-Acosta et al., Mol Cell Neurosci. 2020:103498.

### Immunohistochemical Analysis (IHC)

Coronal sections, 5 μm thickness, of paraffin embedded tissue were placed onto Superfrost Plus microscope glass slides (Thermo Scientific) and were let dry at room temperature (RT) overnight to remove residual water. Sections were de-paraffinized by washing in xylene and re-hydrated in decreasing concentrations of ethanol (from 99% to 70%). Sections were pressure boiled in a Decloaking Chamber (Biocare Medical) immersed in DIVA decloaker 1×solution (Biocare Medical, Concord, USA) at 110 °C for 30 min, or incubated in a water bath heated at 95 °C for 30 min. Slides were let cool down at RT for 20 min, then washed with PBS buffer containing 0.1% tween 20 (PBST) and incubated with peroxidase blocking solution (Dako) for 5 min. The sections were washed in Tris-buffered saline (TBS) and additional blocking was performed with Background punisher (Biocare) for 10 min. Primary antibodies diluted in DAKO (Agilent) antibody diluent were incubated for 45 min at RT. Slides were then washed in TBS and incubated with Mach 2 Double stain 2 containing alkaline phospatase (AP) conjugated secondary anti-rabbit antibody for 30 min at RT. AP staining was detected with permanent red (Biosite). Sections were counterstained with hematoxylin (Mayer), de-hydratated through ethanol (from 70% to 99%), cleared in xylene and mounted with DEPEX mounting media (Merck).

### Microscopy

Stained sections were visualized with a Nikon Eclipse E800M optical microscope with Plan-Apochromate objective of 10× and 20× magnifications. A Nikon fluorescence microscope was used to detect rh Bri2 BRICHOS-mCherry proteins in brain samples and recorded with a 20× objective.

### Western Blotting

Brain tissue from one mouse treated with rh Bri2 BRICHOS-AU1+microbubbles and one negative control were homogenized in 50 mM Tris-HCl (pH 7.4), 150 mM NaCl, 1% (v/v) Triton X-100, 0.1% (w/v) SDS and 10 mM EDTA supplemented with protease inhibitor cocktail (Roche, Indianapolis, IN). Homogenates were centrifuged at 14,000 rpm (20,800 xg) for 30 min at 4 °C, and the supernatant was collected and stored at −20 °C. Protein concentration was measured by BCA method. The homogenates were diluted in homogenization buffer and 1×SDS reducing buffer (containing 2-mercaptoethanol) so that 100 μg total protein were loaded per sample and well. The samples were heated at 97 °C for 10 min and separated on 4-20% precast polyacrylamide gels (Bio-Rad) and blotted on a nitrocellulose (GE Healthcare) membrane. After blotting, the membranes were blocked using 5% skim milk prepared in 0.1% Tween/TBS for 1 h at RT. Thereafter they were rinsed with 0.1% Tween/TBS and primary antibody diluted in 0.1% Tween/TBS was added over night at 4 °C. The membranes were washed three times with 0.1% Tween/TBS and then incubated with secondary antibody prepared in 0.1% Tween/TBS for 1 h at RT. After washing away unbound secondary antibody with 0.1% Tween/TBS, images were acquired using a fluorescence imaging system (Li-Cor, Odyssey CLx).

### Sandwich ELISA

96-well plates (Nunc MicroWell™) were coated with anti Bri2 BRICHOS capture antibody diluted in coating buffer (50 mM carbonate pH 9.6) and incubated overnight at 4 °C. After washing three times in 0.05% Tween/PBS, 1% BSA/PBS was used for blocking for 1 hour. Following the washing and blocking steps, the brain samples (250 μg/ml) and standards diluted in 0.05% Tween/PBS were incubated for 2 hours at RT. The plates were washed and rabbit anti-AU1 primary antibody diluted in 0.05% Tween/PBS was added over night at 4 °C. The plates were washed three times and secondary anti-rabbit antibody diluted in 0.05% Tween/PBS was added for 2 hours at RT. After washing, tetramethylbenzidine (TMB) (Thermo Fisher) solution was added and incubated for 30 min in darkness. The reaction was stopped by adding stop solution for TMB substrates (Thermo Fisher) and absorbance was measured at 450 nm, using the values for brain homogenates from the non-treated mouse as blank. The standard curve was obtained from rh Bri2 BRICHOS-AU1 protein ranging from 0.1 to 64 ng.

Calculation of concentrations and total amounts of rh Bri2 BRICHOS-AU1 in each brain hemisphere was assessed by assuming a brain density of 1.04 mg/ml, an average brain hemisphere weight of 225 mg and average of total protein in brain hemisphere homogenates of 25.6 mg.

### Results

Passage of Rh Bri2 BRICHOS, but not proSP-C BRICHOS Over the BBB in the Non-Sonicated Hemisphere in the Presence of Lipid Microbubbles

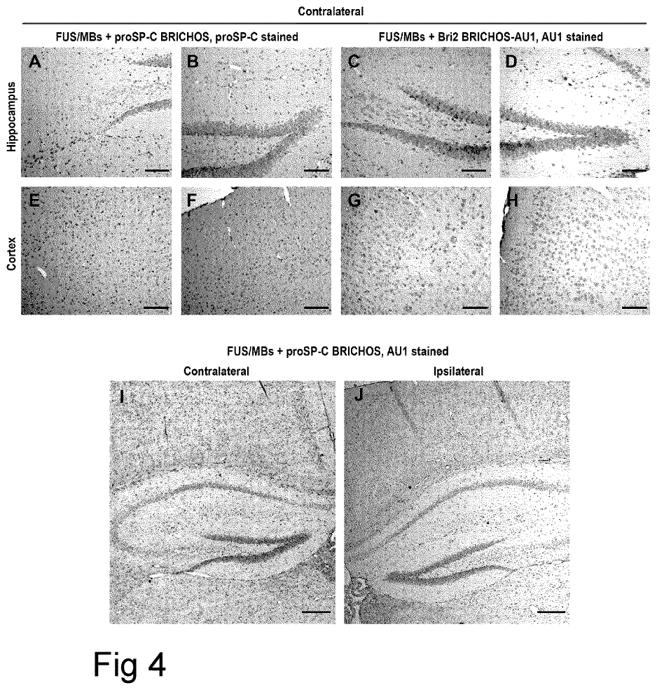

The presence of rh Bri2 BRICHOS-AU1 (SEQ ID NO: 11) in the brain parenchyma was studied. FIG. 4 demonstrates that rh Bri2 BRICHOS, but not rh proSP-C BRICHOS, is detected in non-FUS-targeted brain hemisphere after i.v. injection together with lipid microbubbles. Immunohistochemistry of cortical and hippocampal sections from mice treated with rh proSP-C BRICHOS+microbubbles (A-B, E-F, I-J) or rh Bri2 BRICHOS-AU1+microbubbles (C-D, G-H), from contralateral, non FUS-targeted hemisphere (A-H). (I-J) controls showing no staining for rh Bri2 BRICHOS-AU1 in contralateral or ipsilateral sides of mice treated with intravenous proSP-C BRICHOS+microbubbles and FUS. Tissues were stained using a rabbit anti-proSP-C antibody (A-B, E-F) or a rabbit anti-AU1 antibody (C-D, G-H) followed by AP conjugated secondary antibody and developed with permanent red AP solution. All samples were counterstained with hematoxylin. Scale bars are 100 μm in A-H, and 200 μm in I-J.

Rh Bri2 BRICHOS-AU1 was thus found in cortex and hippocampus of the non FUS-targeted, contralateral hemisphere 2 hours after i.v. administration of 10 mg/kg of rh Bri2 BRICHOS-AU1 in the presence of microbubbles to a wild type mouse (FIG. 4C-D, G-H). Rh proSP-C BRICHOS could not be observed in the non-sonicated hemisphere 2 hours after i.v. injection of 10 mg/kg rh proSP-C BRICHOS and microbubbles in a FUS treated wild type mouse (FIG. 4A-B, E-F). Immunohistochemical staining was negative for rh Bri2 BRICHOS-AU1 in brain sections of a mouse treated with FUS+microbubbles and administered with 10 mg/kg of rh proSP-C BRICHOS, 2 hours after injections, showing that immunoreactivity with anti-AU1 antibody is not an artefact caused by injection of any recombinant protein (FIG. 4I-J).

### Microbubbles Increase BBB Permeability of Rh Bri2 BRICHOS in Wild Type Mice

The effects of microbubbles alone, i.e. without any application of FUS, on rh Bri2 BRICHOS permeability over the BBB were further studied by administering rh Bri2 BRICHOS domain with either an AU1 tag (rh Bri2 BRICHOS-AU1; SEQ ID NO: 11) or mCherry protein (rh Bri2 BRICHOS-mCherry; SEQ ID NO: 12) genetically linked to the C-terminus. SonoVue® microbubbles (dose given 5 μl/g body weight) and different doses of rh Bri2 BRICHOS-AU1 were individually injected into the lateral tail vein of adult wild type mice, and after 2 hours brains were perfused and collected for analyses. Control mice were administered with SonoVue® microbubbles and PBS. The presence of rh Bri2 BRICHOS-AU1 in the brain parenchyma was studied by immunohistochemistry and western blot, while the amounts of rh Bri2 BRICHOS-AU1 were assessed by sandwich ELISA (FIG. 3A).

In order to study the effects of microbubbles on BBB permeability of rh Bri2 BRICHOS fused to a folded globular protein, rh Bri2 BRICHOS-mCherry (44 kDa total molecular weight) was chosen as it can be detected by the fluorescence properties of folded mCherry, which remain unchanged also in fusion with rh Bri2 BRICHOS. In this case, i.v. injections of SonoVue® and 20 mg/kg of rh Bri2 BRICHOS-mCherry (SEQ ID NO: 12) was applied to two wild type mice, while control mice received rh Bri2 BRICHOS-mCherry only (FIG. 3B). Brains were collected 2 hours after injections and analysed macroscopically and under a fluorescence microscope.

The inventors have surprisingly found that microbubbles alone increase passage of rh Bri2 BRICHOS-AU1 over the BBB.

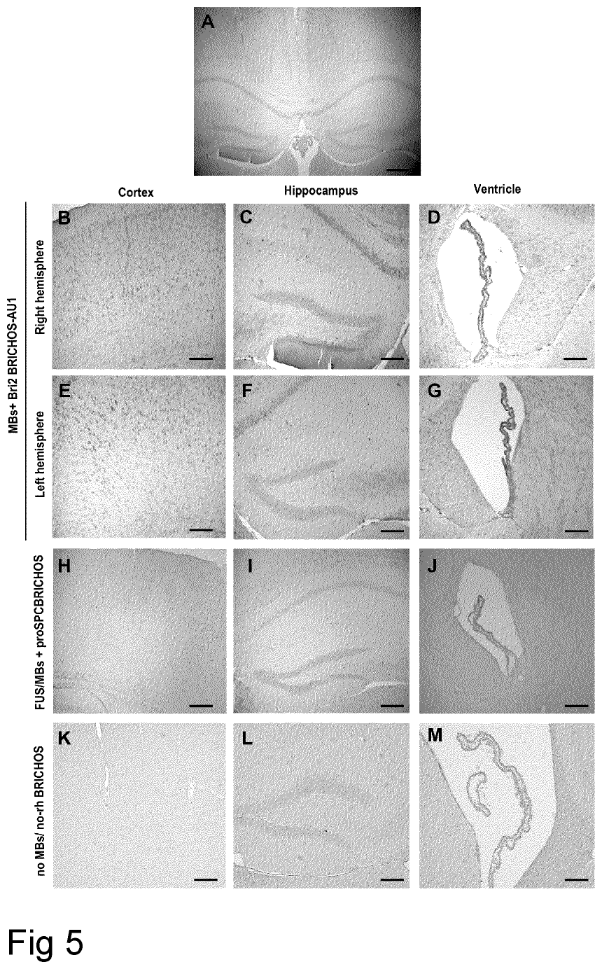

FIG. 5 shows that Bri2 BRICHOS is detected in both hemispheres after i.v. injection together with lipid microbubbles. (A-G) Images from cortex, hippocampus and lateral ventricles from a mouse treated with microbubbles+mg/kg rh Bri2 BRICHOS-AU1. (H-J) Images from a mouse injected with 10 mg/kg rh proSP-C BRICHOS and microbubbles. (K-M) Images from a control mouse that received no injections. All sections were incubated with an anti-AU1 antibody followed by an anti-rabbit antibody conjugated with AP and 10 developed with permanent red AP solution, and counterstained with hematoxylin. Sizes of scale bars are 500 μm in panel A and 200 μm in panels B-M.

Rh Bri2 BRICHOS-AU1 was detected in the cortex, hippocampus and in the choroid plexus in the lateral ventricles by immunohistochemistry using an antibody against the AU1 tag, after i.v. injection of microbubbles and rh Bri2 BRICHOS-AU1 at the dose of 10 mg/kg (FIG. 5A-G). No differences in staining intensity were seen between the two hemispheres (FIG. 5A-G).

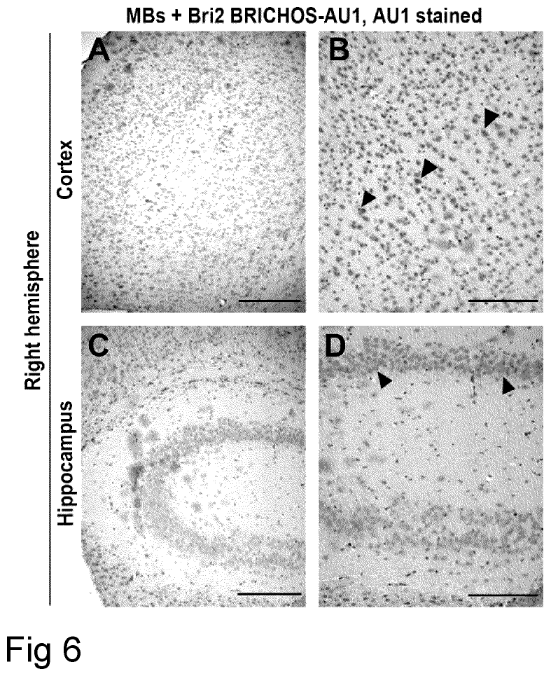

FIG. 6 demonstrates that intracellular immunostaining for rh Bri2 BRICHOS-AU1 is observed in the cortex and hippocampus after i.v. injection together with lipid microbubbles. Images show cortex (A,B) and hippocampus (C,D) of the right hemisphere from a mouse treated with microbubbles+10 mg/kg rh Bri2 BRICHOS-AU1 (SEQ ID NO: 11). Sections were stained for rh Bri2 BRICHOS-AU1 using anti-AU1 antibody and counterstained with hematoxylin. The arrowheads in (B) and (D) panels indicate intracellular staining for rh Bri2 BRICHOS-AU1. Sizes of scale bars are 100 μm for panels A, C; and 50 μm for panels B,D.

Intracellular staining was thus observed in cells present in the cortex and in the hippocampus (FIG. 6A-D). Control brains from mice either injected with rh proSP-C BRICHOS or with PBS were stained in the same way with the anti-AU1 antibody but showed no immunoreactivity (FIG. 5H-M).

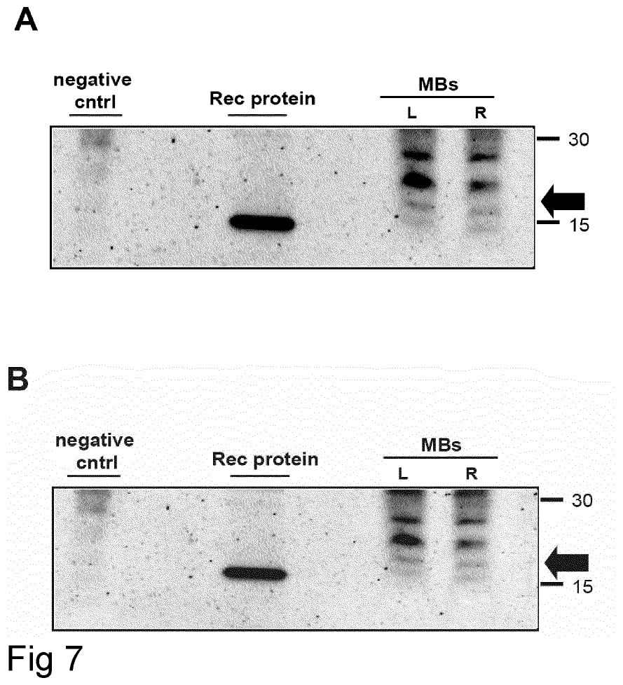

Furthermore, homogenates of both hemispheres from the mouse administered with microbubbles plus 10 mg/kg rh Bri2 BRICHOS-AU1 were analysed by western blot using both anti-AU1 and anti-Bri2 antibodies, which revealed bands migrating as rh Bri2 BRICHOS-AU1 and somewhat slower, while the corresponding bands were absent in a control sample. FIG. 7 shows western blot detection of rh Bri2 BRICHOS-AU1 (SEQ ID NO: 11) in both brain hemispheres after systemic injection together with lipid microbubbles. Western blot analysis of left (L) and right (R) hemispheres from one mouse collected two hours after i.v. injection of microbubbles+rh Bri2 BRICHOS-AU1, and a brain homogenate from a non-treated control mouse (negative control). Rh Bri2 BRICHOS-AU1 is detected using anti-AU1 antibody (A) and an anti-Bri2 antibody (B), as indicated by arrows on the right side of the gel. Lanes marked Rec protein contain 5 ng of rh Bri2 BRICHOS-AU1.

In agreement with immunohistochemistry and western blot results, analyses of homogenates of both hemispheres of the microbubbles plus 10 mg/kg rh Bri2 BRICHOS-AU1 treated mouse 2 hours after injection by sandwich ELISA showed that the rh Bri2 BRICHOS-AU1 concentration in the left hemisphere was 390 nM while in the right hemisphere it was 250 nM, which together correspond to 1% of the total amount administered. Interestingly, higher amounts of isolated recombinant Bri2 BRICHOS-AU1 had reached the brain parenchyma after administration with lipid microbubbles, without ultrasound treatment, compared to without administration of microbubbles. About 1% of the total isolated recombinant Bri2 BRICHOS-AU1, administered at a dose of 10 mg/kg together with lipid microbubbles was detected in the brain 2 hours after intravenous injection, while between 0.1 and 1% (mean 0.5%) was detected after administration of isolated recombinant Bri2 BRICHOS-AU1 at a dose of 20 mg/kg without microbubbles. This is surprising because microbubbles alone (without ultrasound treatment) have not yet been described to increase passage of a protein over the BBB or affect uptake into neurons.

The inventors have furthermore found that rh Bri2 BRICHOS-mCherry passes into the brain parenchyma of wild type mice after injection with, but not without, lipid microbubbles. The BBB permeability of rh Bri2 BRICHOS-mCherry in wild type mice injected with rh Bri2 BRICHOS-mCherry with or without microbubbles was evaluated by observing brains macroscopically and brain sections under a fluorescence microscope.

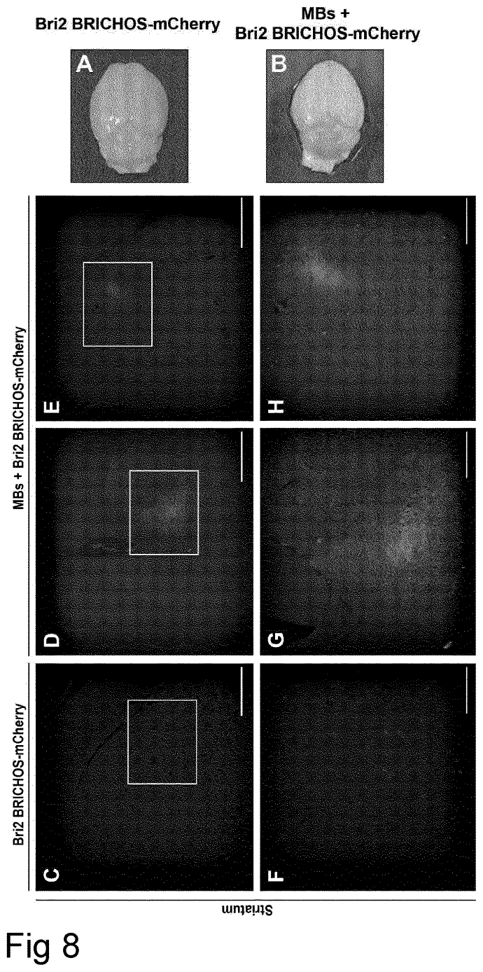

FIG. 8 shows that Rh Bri2 BRICHOS-mCherry (SEQ ID NO: 12) is found in the striatum after systemic injection in combination with lipid microbubbles. (A-B) Photographs of mouse brains after injection of rh Bri2 BRICHOS-mCherry only (A) or in combination with microbubbles (B). (C—H) Fluorescence of striatum regions collected 2 hours after injection of (C,F) 20 mg/kg rh Bri2 BRICHOS-mCherry only, and (D-E, G-H) microbubbles and 20 mg/kg rh Bri2 BRICHOS-mCherry. Sizes of scale bars are 200 μm for panels C-E, and 100 μm for panels F-H. Areas marked with white rectangles in C, D, E panels are enlarged in the F, G,H panels, respectively.

The brains of thoroughly perfused mice injected with either rh Bri2 BRICHOS-mCherry alone (one mouse) or microbubbles and rh Bri2 BRICHOS-mCherry (two mice) collected 2 hours after injections revealed a clear colour (white colour in FIG. 8) in the outer layer of the cortex and in the cerebellum only in the mice with rh Bri2 BRICHOS-mCherry plus microbubbles (FIG. 8A, B). Moreover, fluorescence (white colour in FIG. 8) was detected in the striatum region in paraffin embedded sections of the two mice injected with microbubbles and rh Bri2 BRICHOS-mCherry (FIG. 8D, E, G, H) but was not seen the mouse injected with rh Bri2 BRICHOS-mCherry alone (FIG. 8C, F). The data strongly suggests that intravenously injected rh Bri2 BRICHOS-mCherry passes into the brain parenchyma when microbubbles are injected into the circulation.

Further experiments with administration of Bri2 BRICHOS

### Methods

### Cell Culture

hCMEC/D3 cells (MilliporeSigma, Burlington, MA) were cultivated in EndoGRO-MV Complete Media (MilliporeSigma, Burlington, MA), supplemented with 1 ng/ml recombinant human fibroblast growth factor basic (FGF-2, Gibco, Invitrogen, Waltham, MA) and penicillin-streptomycin (100 IU/ml, 100 μg/ml, Gibco, Thermo Fisher Scientific, Waltham, MA) in T75 tissue culture flasks (TC-coated, DeltaLab, Spain) coated with 5-10 μg/cm² rat tail collagen type I (MilliporeSigma, Burlington, MA). The human brain microvascular endothelial cell line hCMEC/D3 is used as a human blood-brain barrier (BBB) in vitro model which produces an accurate in vivo phenotype, is reproducible and simply maintained. The hCMEC/D3 cell line has preserved the expression of majority of the transporters and receptors inherent to the BBB and therefore is efficiently used for studying the transport kinetics and mechanisms over the BBB. The hCMEC/D3 cell line has been previously described for the transport across the BBB for Aβ40 peptide, transthyretin and for several drug candidates.

Human neuroblastoma (SH-SY5Y) cell line (ATCC, Manassas, VA) was grown in Dulbecco's Modified Eagle's Medium (DMEM, PAN-Biotech, Germany) with 4.5 g/L glucose, glutamine (2 mM) and 3.7 g/L NaHCO₃, supplemented with 10% Fetal Bovine Serum (FBS, Sera Plus, PAN-Biotech, Germany) and penicillin-streptomycin (100 IU/ml, 100 μg/ml) in TC-coated T75 tissue culture flasks. The human neuroblastoma cell line SH-SY5Y is a thrice-subcloned cell line derived from the SK—N-SH neuroblastoma cell line. It is widely used an in vitro neuronal model for studies of neurodegenerative diseases.

Immortalized human astrocyte cell line (Innoprot, Spain) was grown in astrocyte medium (Innoprot, Spain), containing 2% FBS, 1% astrocyte growth supplement and 1% penicillin-streptomycin (100 IU/ml, 100 μg/ml) in T75 tissue culture flasks coated with 2 μg/cm² poly-L-lysine (Sigma-Aldrich, St. Louis, MO). Human embryonic kidney 293 (HEK-293) cell line (ATCC, Manassas, VA) was grown in Eagle's Minimum Essential Medium (EMEM, ATCC, Manassas, VA), containing 2 mM L-glutamine, 1 mM sodium pyruvate and 1.5 g/L NaHCO₃, supplemented with 10% FBS and penicillin-streptomycin (100 IU/ml, 100 μg/ml) in TC-coated T75 tissue culture flasks. Cultures were maintained in a humidified atmosphere (5% CO2/95% air) at 37 °C and media was changed every other day. At approximately 80% confluency cells were trypsinated (0.025% trypsin, 0.01% EDTA, Gibco, Thermo Fisher Scientific, Waltham, MA) and passaged until passage 35 for the hCMEC/D3, human astrocytes and SH-SY5Y cell lines.

Human embryonic kidney 293 (HEK-293) cell line (ATCC, Manassas, VA) was grown in Eagle's Minimum Essential Medium (EMEM, ATCC, Manassas, VA), containing 2 mM L-glutamine, 1 mM sodium pyruvate and 1.5 g/L NaHCO₃, supplemented with 10% FBS and penicillin-streptomycin (100 IU/ml, 100 μg/ml) in TC-coated T75 tissue culture flasks.

Cultures were maintained in a humidified atmosphere (5% CO₂/95% air) at 37 °C and media was changed every other day. At approximately 80% confluency cells were trypsinated (0.025% trypsin, 0.01% EDTA, Gibco, Thermo Fisher Scientific, Waltham, MA) and passaged (until passage 35 for the hCMEC/D3 and SH-SY5Y cell lines).

hCMEC/D3 Monolayer Preparation

For the preparation of the hCMEC/D3 cell monolayers, transwell ethylene terephthalate (PET) membrane inserts (24-well, clear, pore size 0.4 μm, CellQART, SABEU GmbH & Co. KG, Germany) were coated with rat tail type I collagen (150 μg/ml in 1×Dulbecco's Phosphate-buffered saline pH 7.4, 1×DPBS, Gibco, Thermo Fisher Scientific, Waltham, MA) for 2 h at 37 °C. Excess collagen was removed and the inserts were washed twice with 1×DPBS. hCMEC/D3 cells were seeded onto the freshly prepared collagen-coated inserts at a density of 50,000 cells/cm² and maintained in a humidified atmosphere (5% CO₂/95% air) at 37 °C. Media was changed every second day and the cell monolayer was established by day 6.

Analysis of hCMEC/D3 Monolayer Integrity

The integrity of the monolayer was analyzed by crystal violet staining. The expression of the tight junction proteins zonula occludens-1 (ZO-1), junctional adhesion molecule A (JAM-A) and claudin-5 was confirmed by western blot and immunostaining. For crystal violet staining the cells were fixed with 3.7% formaldehyde (Sigma-Aldrich, St. Louis, MO) at room temperature for 2 min and permeabilized with 100% methanol (Sigma-Aldrich, St. Louis, MO) at room temperature for 20 min, after which the cells were stained with 0.01% crystal violet (Sigma-Adrich, St. Louis, MO) in 2% ethanol and analyzed using a fluorescence microscope (Axioskop 40, Zeiss, Germany). Images were captured by fluorescence microscopy using green (FITC/488) and red (TexasRed/570) channels.

For determination of the tight junction proteins, hCMEC/D3 cells were seeded onto collagen-coated 12-well plates (TC-coated, Cellstar, Greiner Bio-One, Austria) with at a seeding density of 50,000 cells/cm² and maintained in a humidified atmosphere (5% CO₂/95% air) at 37 °C, changing the media every second day. After 6 days the cells were removed from the plate by scraping in ice cold 1×PBS, centrifuged at 14,000×g, 4 °C for 10 min, lysed in radioimmunoprecipitation assay (RIPA) buffer (Sigma, St. Louis, MO) containing 1 mM phenylmethylsulfonyl fluoride (PMSF, Thermo Scientific, Waltham, MA) and 1×Complete Protease Inhibitor (Roche, Switzerland), and then sonicated at 50% amplitude for 10 sec and incubated on ice for 20 min. Cell lysates were cleared by centrifugation at 14,000×g, 4 °C for 15 min and the total amount of protein in the cell lysates was determined by Bicinchoninic acid (BCA) assay (Pierce, Thermo Fisher Scientific, Waltham, MA), following manufacturers protocol. For western blots (see below) anti-Claudin-5, anti-JAM-A and anti-ZO-1 polyclonal rabbit primary antibodies (Invitrogen, Thermo Fisher Scientific, Waltham, MA), were used at 1:1000 dilution.

hCMEC/D3 and Human Astrocyte Coculture Preparation

For the direct coculture model the PET membrane on the apical side of the cell culture inserts were first coated with rat tail type I collagen (see hCMEC/D3 monolayer preparation). The inserts were flipped and the basolateral side of the cell culture insert was coated with poly-L-lysine (2 μg/cm² in ddH₂O) overnight at 37 °C. Excess poly-L-lysine was removed and the membrane was washed twice with ddH₂O. Human astrocytes were plated on the basolateral side of the cell culture insert at 20,000 cells/cm² density and left to attach for 4 h at 37 °C in upside-down orientation, after which the inserts were placed in the normal orientation and grown for 48 h in astrocyte medium. The hCMEC/D3 cells were seeded on the apical side of the insert at 50,000 cell/cm² density. The hCMEC/D3 cells were left to proliferate in the EndoGro complete medium in the apical compartment and the human astrocytes in the astrocyte medium in the basolateral compartment.

For the indirect coculture model the 24-well plates were covered with poly-L-lysine similarly as cell culture insert membranes. The human astrocytes were seeded at 20,000 cells/cm² density and incubated for 48 h. Cell culture inserts were coated with rat tail type I on the apical side and hCMEC/D3 cells were seeded similarly to the direct coculture model. The hCMEC/D3 coated inserts were incubated separately in EndoGro complete medium overnight at 37 °C and then inserted to the 24-well plates containing human astrocytes in astrocyte medium.

Media was changed every second day and FITC-dextran permeability and TEER values were measured on days 3-9 after seeding, similarly to the hCMEC/D3 monolayer model.

Analysis of hCMEC/D3 Monolayer Permeability

Experiments were conducted on culture day 6 by adding different BRICHOS protein constructs, or relevant controls to the apical side of the hCMEC/D3 monolayer. BRICHOS constructs used were rh proSP-C BRICHOS wt, rh proSP-C BRICHOS T187R, rh Bri2 BRICHOS wt monomers and oligomers, rh Bri2 BRICHOS R221E monomers, S-tag-Bri2 BRICHOS monomers and oligomers, mCherry-Bri2 BRICHOS monomers, NT-Bri2 BRICHOS monomers and oligomers and an unresolved mixture of different S-tag-Bri3 BRICHOS assembly states. Controls used were mCherry, NT*-tag, rh Aβ42 and apolipoprotein A-I (purified from human plasma with >98% purity; Chemicon, Sigma-Aldrich, St. Louis, MO). Concentrations used were 1 μM, 0.5 μM, 0.25 μM, 0.1 μM or 0.05 μM; and incubation times were either 2 h or 24 h. Monolayers were incubated in a humidified atmosphere (5% CO₂/95% air) at 37 °C. After the incubation, medium on the apical and basolateral sides of the monolayer was removed and analyzed by Western blotting and the band intensities measured by ImageJ. For the western blot (see below) anti-Bri2 BRICHOS primary antibody (produced in goat and purified in house) in 1:1000 dilution, anti-proSP-C BRICHOS primary antibody (produced in goat and purified in house) at 1:2000 dilution, anti-S-tag primary antibody (S-tag protein HRP conjuc. Novagen, Merck, Germany) at 1:5000 dilution, anti-NT primary antibody (produced in rabbit and purified in house at 1:5000 dilution, anti-RFP primary antibody (produced in mouse, Thermo Fisher Scientific, Waltham, USA) at 1:1000 dilution and anti-human Apolipoprotein A-I primary antibody (produced in goat, Calbiochem, San Diego, CA) were used. For mCherry and mCherry-Bri2 BRICHOS monomers, fluorescence intensity of the apical and basolateral medium was recorded at λₑₓ 570 nm and λₑₘ 610 nm, after which the cells were fixed and analyzed by fluorescence microscopy to analyse the uptake of mCherry or mCherry-Bri2 BRICHOS into cells.

The permeability of the monolayer was also analyzed for fluorescein isothiocyanate dextran (FITC-dextran, Thermo Fisher Scientific, Waltham,

MA) as negative control and human recombinant insulin (Sigma-Aldrich, St. Louis, MO) as positive control. For determining the FITC-dextran, insulin and propidium iodide permeability, 1 μM solutions in cell culture media were applied to the apical side of the monolayer and incubated in a humidified atmosphere (5% CO₂/95% air) at 37 °C for 24 h, after which the media from the apical and basolateral sides was removed. FITC fluorescence was measured at λₑₓ 492 nm and λₑₘ 518 nm. Insulin permeability was determined by western blot and an anti-Insulin monoclonal primary antibody (Sigma, St. Louis, MO) at 1:1000 dilution.

### Cell Viability

Cell viability was assessed by tetrazolium-based Thiazolyl Blue Tetrazolium Bromide (MTT) assay. For the MTT assay, 50 000 cells/cm² were seeded onto clear 96-well plates (TC, Cellstar, Greiner Bio-One, Austria) and grown until confluent, after which the cells were treated with the rh BRICHOS domains in 8 replicates for 24 h in a humidified atmosphere (5% CO₂/95% air) at 37 °C. Treated cells were incubated with 0.5 mg/ml MTT (Sigma-Aldrich, St. Louis, MO) solution in FBS-free cell media for 4 h in a humidified atmosphere (5% CO²/95% air) at 37 °C. The MTT solution was removed and the formazan crystals were dissolved in Dimethyl sulfoxide (DMSO, Sigma-Aldrich, St. Louis, MO) and incubated with shaking for 15 min. Absorbance was measured at 570 nm using a FLUOstar OPTIMA microplate reader (BMG Lab Tech, Germany). Untreated cells were used as positive control, and media without cells was used as a negative control. Also 2 μM Staurosporine from Streptomyces sp. (SP, Sigma-Aldrich, St. Louis, MO) was used as a negative control.

### Cell Uptake

For the cell uptake analysis, hCMEC/D3 cells were seeded onto collagen-coated 12-well plates and non-differentiated SH-SY5Y cells were seeded onto 12-well plates without collagen treatment, at a seeding density of 75,000 cells/cm² and maintained in a humidified atmosphere (5% CO₂/95% air) at 37 °C, changing the media every second day. Once confluent, the hCMEC/D3 cells were treated with 1 μM rh Bri2 BRICHOS monomers, oligomers or proSP-C monomers for 2 h and 24 h. The SH-SY5Y cells were treated with either rh Bri2 BRICHOS monomers in concentrations 1 μM, 0.5 μM, 0.25 μM, 0.1 μM, 0.05 μM for 24 h; rh Bri2 BRICHOS monomers in concentrations 1 μM and 0.25 μM for 2 h and 24 h; rh Bri2 BRICHOS oligomers in 1 μM concentration for 24 h; or 0.25 μM and 1 μM rh proSP-C for 2 h and 24 h. Untreated cells were used as control. After the treatment, the cells were lysed as described for the HEK-293 cells. For the Western blot (see below), anti-Bri2 BRICHOS primary antibody (produced in goat and purified in house) was used at 1:1000 dilution, and anti-proSP-C BRICHOS primary antibody (produced in goat and purified in house) was used at 1:2000 dilution.

### Transcytosis Inhibition

The monolayer and cellular uptake experiments were done essentially as described above, but after the monolayer had formed, it was serum starved for 4 h in FBS-free EndoGro medium. Then the monolayer was incubated with 30 μM Dyngo-4a (Selleck Chemicals, Boston, MA) for 20 min in FBS-free medium. Afterwards, the monolayer permeability assay was conducted with 1 μM rh Bri2 BRICHOS R221E monomers in FBS-free EndoGro medium together with 30 μM Dyngo-4a, for 24 h in a humidified atmosphere (5% CO₂/95% air) at 37 °C. Control experiments without Dyngo-4a were conducted similarly in serum-free medium. The apical and basolateral sides were analyzed by western blot for determination of net average transport over the monolayer. For the cellular uptake, cells were similarly serum starved in FBS-free EndoGro Medium for 4 h and incubated with 30 μM Dyngo-4a for 20 min. Uptake of 1 μM rh Bri2 BRICHOS R221E monomers in FBS-free EndoGro medium was analyzed after 4 h and 24 h in the presence of 30 μM Dyngo-4a. Control experiments without Dyngo-4a were conducted similarly in serum-free medium. Cell medium and lysates were analyzed by western blot.

### Western Blot

Samples were separated by Tris-glycine SDS-PAGE, using 12% separating gel, under reducing conditions, including 5% β-Mercaptoethanol (Sigma, St. Louis, MO) and 5 mM DTT in the sample buffer and were heated at 95 °C for 5 min. Spectra Multicolor Broad Range protein ladder was used (Thermo Fisher Scientific, Waltham, USA). For the monolayer permeability assessment, 4 μl of protein sample in cell culture media was loaded in the wells. For the cell uptake assessment, 7 μg of cell lysate was loaded.

Electrophoresis was conducted at 120 V, and electrotransfer to a Polyvinylidene fluoride (PVDF) membrane (0.2 μm, Amersham Hybond, GE Healthcare, Chicago, IL) was achieved at 15 V for 30 min (Semi-dry transfer cell, Bio-Rad, Hercules, CA) with a transfer buffer formulation of 25 mM Tris-HCl pH 8.3, 192 mM glycine, 0.05% SDS, 20% methanol.

The membranes were soaked in blocking buffer [5% nonfat dry milk (AppliChem, Germany) diluted in 1×PBS containing 0.1% Tween-20 (Sigma, St. Louis, MO) (1×PBS-T)] for 30 min at room temperature and incubated with the primary antibody overnight at 4 °C. Membranes were washed 3 times with 0.5% nonfat dry milk in 1×TPBS-T and incubated with horseradish peroxidase conjugated secondary antibody (Goat Anti-Rabbit IgG from abcam, UK in 1:3000 dilution; Rabbit Anti-Goat IgG from Invitrogen, Waltham, MA at 1:10000 dilution; Rabbit Anti-Mouse IgG from Invitrogen, Waltham, Ma at 1:10000 dilution; Goat Anti-Human IgG Fc from Invitrogen, Waltham, Ma in 1:1000 dilution) for 1 h at room temperature. Membranes were washed again as above, and also twice with 1×PBS. Blots were developed using chemiluminescent detection system (Pierce ECL plus, Thermo Fisher Scientific, Waltham, USA). For cell lysates the measurements were normalized by β-actin (0.01 μg/ml) (R&D Systems, Minneapolis, MI) after mild stripping (0.2 M glycine, 0.1% SDS, 1% Tween-20 pH 2.2). The intensities were assessed using ImageJ software.

For insulin and NT*, extra experimental procedures were added. The blotted membrane was soaked with blocking buffer [1% nonfat dry milk and 0.1% BSA in TBS-T (TBS [50 mM Tris-HCl at pH 7.4, 150 mM NaCl] containing 0.1% Tween 20)] for 5 min and washed with PBS-T for 3 min. The membrane was incubated in 0.2% Glutaraldehyde in PBS-T for 15 min and washed 3 times with PBS-T, after which it was immersed in citrate retrieval buffer (10 mM citric acid pH 6.0, 1 mM EDTA, 0.05% Tween 20) and microwaved for 10 min at 600 W after boiling. After cooling to room temperature, the membrane was soaked with quenching buffer (200 mM glycine in PBS-T) for 10 min. After these extra steps, blotting was performed as described above.

### Analysis of NT-BRICHOS Fusion Protein Passage Over BBB in Wildtype Mice

A gene fragment encoding rh NT*-Bri2 BRICHOS fusion protein, where NT* is a solubility tag (as described in WO 2017/081239 A1) followed by human Bri2 residues 113-231, was cloned and expressed. The protein was expressed in Shuffle T7 competent E. coli cells that were grown in Lysogeny Broth (LB) medium supplemented with 15 μg/mL kanamycin at 30 °C. When OD_{600 nm} reached ~0.9, the temperature was lowered to 20 °C and over-night protein expression was induced by addition of 0.5 mM isopropyl b-D-1-thiogalactopyranoside (IPTG). Cells were harvested by centrifugation (3,000×g, 4 °C) and cell pellets were re-suspended in 20 mM Tris-HCl pH 8.0 followed by 5 min sonication (2 s on 2 s off, 65% power, on ice). The lysate was centrifuged (24,000×g, 4 °C) for 30 min, and the supernatant containing the target protein was then purified with an immobilized metal affinity chromatography (IMAC) column (Ni Sepharose™ 6 Fast Flow; GE Healthcare, UK) equilibrated with 20 mM Tris-HCl PH 8.0. The fusion protein was eluted with 300 mM imidazole in 20 mM Tris-HC pH 8.0, and dialyzed (regenerated cellulose RC, 6-8 kDa membrane; Spectrum Lab) against 20 mM Tris-HCl pH 8.0 overnight in cold room.

All the animal handling and experimental procedures were carried out in the animal facility, Huddinge campus, Karolinska Institutet according to local ethical guidelines and approved by Södra Stockholm's Djurförsöksetiska Nämnd (dnr S 6-15) and Linköping's animal ethical board (ID 855). Three months old C57BL/6NTac (Taconic, Denmark) mice and 11 months old C57BL/6J mice (Janvier labs, France) were kept under controlled humidity and temperature on a 12-hour light-dark cycle, group-housed (seven per cage) with food and water available ad libitum. 3 Mice received a single i.v. injection of NT*-Bri2 BRICHOS fusion protein, 10 mg/kg, or equal volume of PBS, into the lateral tail vein by using a 0.3 ml syringe with a 30-gauge needle. Before the injections the mice were placed in a single cage under a heat lamp for 5 min, to dilate the tail veins. The 3 months old mice were anesthetized with isoflurane and intracardially perfused with 40 mL of saline (0.9% NaCl) 2 h after the injections. 11 months old mice received single i.v. injections of NT*-Bri2 BRICHOS fusion protein 10 mg/kg, or an equal volume of PBS, anesthetized and perfused 2 h after the injections. Brains were quickly removed and snap-frozen in dry ice and stored at −80 °C until analysis.

Immunohistochemical staining for NT*-Bri2 BRICHOS fusion protein was performed in 5 μm thick coronal sections of paraffin-embedded mouse brain tissue. The sections were de-paraffinized in xylene and re-hydrated in graded alcohol series from 99% to 70%. Brain sections were pre-treated for antigen retrieval in DIVA Decloaker 1×solution (Biocare Medical) inside a pressure cooker (Biocare Medical) at 110 °C for 30 min. The slides were cooled down at room temperature (RT) for 30 min, then washed with Tris-buffered saline containing 0.05% Tween 20 (TBS-T) and incubated first with peroxidase blocking solution (Dako) for 5 min. After washing in TBS-T, brain sections were additionally blocked with Background punisher (Dako) for 10 min. Subsequently, the slides were incubated with a solution containing the primary anti-NT antiserum from rabbit diluted in DAKO (Agilent) antibody diluent, overnight at 4 °C. After washing with TBS-T, the sections were incubated for 30 min at RT with secondary cocktail containing horseradish peroxidase (HRP)-conjugated goat anti-rabbit antibody. HRP immunoreactivity was detected by permanent green (Biosite) solution. The sections were counterstained in hematoxylin Mayer, de-hydrated, cleared in xylene and mounted with DEPEX mounting media (Merck). Images were acquired using a Nikon Eclipse E800 light microscope linked to a high-resolution camera and using a 20× objective and 40× objective.

### Results

Bri2 BRICHOS Accumulates in CNS after Intravenous Administration

Aβ precursor protein (App) knock-in mouse models (Saito T. et al. (2014) Nat Neurosci. 17, 661-663), both App^{NL-G-F} mice (that express human Aβ with the Arctic mutation) and App^{NL-F} mice (that express wildtype human Aβ) were treated with repeated intravenous injections of rh Bri2 BRICHOS R221E (SEQ ID NO: 13). After the end of 10-12 weeks of treatment, Bri2 BRICHOS amounts in the CNS were analysed.

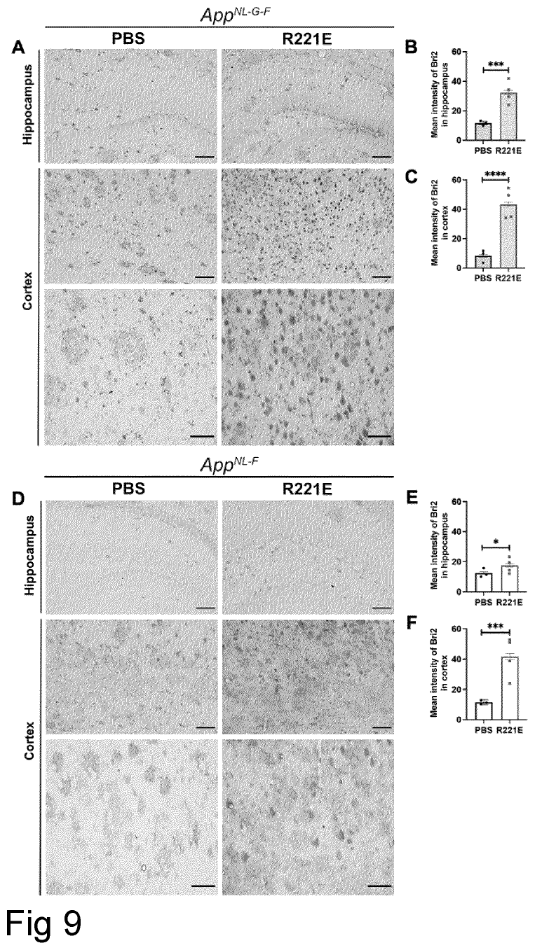

FIG. 9 shows Bri2 BRICHOS in App^{NL-G-F} and App^{NL-F} mouse brains after repeated injections of rh Bri2 BRICHOS R221E. Representative sections (A,D) were double stained with anti-Bri2 BRICHOS antibody (red/pink color) and 82E1 anti-Aβ antibody (green color) counterstained with hematoxylin Mayer (blue color) in rh Bri2 BRICHOS R221E or PBS treated App^{NL-G-F} and App^{NL-F} mice. Histograms represents mean intensity of Bri2 BRICHOS in hippocampus (B,E) and cortex (C,F) of App^{NL-G-F} (B,C) and App^{NL-F} (E,F) mice. Data are shown as mean±SEM (n=3-4 mice/group, 4 histological sections from each mouse analyzed). Unpaired parametric two-tailed t-test was used to calculate p-values. Scale bars represents 400 μm (upper four panels in A and D) and 100 μm (lower two panels in A and D). *p<0.05, **p<0.01, *p<0.001, *p<0.0001.

The rh Bri2 BRICHOS R221E treated App knock-in mice showed more abundant overall Bri2 BRICHOS staining in the brain tissue including in neurons and around the Aβ plaques (FIG. 9, panels A and D) compared to the PBS treated control mice. There is a significant increase in overall Bri2 BRICHOS staining, analyzed from mean intensity of Bri2 BRICHOS between PBS and rh Bri2 BRICHOS R221E treated samples in both App^{NL-G-F} and App^{NL-F} mice (FIG. 9, panels B, C, E, F). Notably, the mice were sacrificed 2-4 weeks after the last dose of rh Bri2 BRICHOS R221E. These results strongly support that intravenously injected rh Bri2 BRICHOS R221E crosses the BBB in App knock-in AD mouse models and stays in the CNS parenchyma and cells for substantial time.

### Cell Viability

The effect of the rh BRICHOS domain on hCMEC/D3 cells and undifferentiated SH-SY5Y cells was assessed after 24 h incubation by tetrazolium-MTT cell viability assay. Rh Bri2 BRICHOS monomers show no toxicity to human brain microvascular endothelial cell line hCMEC/D3 and human neuroblastoma cell line SH-SY5Y, but a slight decrease in the cell mitochondrial activity in the presence of high concentration rh proSP-C BRICHOS and rh Bri2 BRICHOS oligomers or crude solution.

The effect of the rh Bri2 BRICHOS (113-231) wt monomers and oligomers on human cell lines hCMEC/D3 and SH-SY5Y is concentration dependent, with the concentration increase lowering the cell viability. For the rh Bri2 BRICHOS monomers in concentration range 1.0-0.1 μM, the SH-SY5Y cell viability increases from 94.0% to 99% and the hCMEC/D3 viability increases from 100.4% to 106.6%, with the rh Bri2 BRICHOS monomer lower concentrations slightly increasing hCMEC/D3 cell metabolic activity. Rh Bri2 BRICHOS oligomers at 1 μM concentration show 83.5% viability on SH-SY5Y cells and 86.7% viability on hCMEC/D3 cells. Rh proSP-C BRICHOS at 0.5 μM concentration show 88.9% viability on SH-SY5Y cells and at 1 μM concentration 90.3% viability on hCMEC/D3 cells. It is possible for the rh Bri2 BRICHOS oligomers at high concentration, like Aβ peptide, to commence a mild stress in the endoplasmic reticulum (ER) and Ca²⁺ release. In case of rh Bri2 BRICHOS monomers, the integrity of the cell membrane remains similar to the positive control. With the physiological concentration for the BRICHOS domain in human plasma being lower, it can be assumed that the BRICHOS domain is not harmful to the integrity of the BBB in normal conditions.

### Cell Uptake

Cell uptake for the rh BRICHOS domain was assessed for human cell lines SH-SY5Y and hCMEC/D3 after 2 h and 24 h incubation by western blotting and determined by band intensities.

24 h incubation of human neuroblastoma cell line SH-SY5Y with the rh Bri2 BRICHOS monomers in concentration ranging from 0.05 μM to 1 μM show a linear uptake. Bri2 BRICHOS oligomers are detected in the cell lysate to a lesser degree than the Bri2 BRICHOS, which may indicate the oligomers adhering to the cell surface. ProSP-C BRICHOS is similarly taken up by the SH-SY5Y cells, but to a lesser degree. No endogenous BRICHOS domain can be detected in the non-treated cells, showing the detectable BRICHOS domain is taken up from the cell media.

Cell uptake of different rh BRICHOS domain constructs and concentrations after 2 h and 24 h was also studied for the human brain microvascular endothelial cells hCMEC/D3. Similarly to the SH-SY5Y cells, Bri2 BRICHOS monomers and proSP-C BRICHOS are taken up by the hCMEC/D3 cells, but the Bri2 BRICHOS oligomers seem not to be taken up. No endogenous BRICHOS domain can be detected in the non-treated cells, showing the detectable BRICHOS domain is taken up from the cell media.

Rh Bri2 BRICHOS wt monomers passed efficiently also over astrocyte/hCMEC/D3 coculture models. The passage over astrocyte/hCMEC/D3 direct cocultures (7.0%) was somewhat lower than for the astrocyte/hCMEC/D3 indirect cocultures (8.6%) and hCMEC/D3 monocultures (10.5%). This is likely partly related to the higher TEER (transepithelial electric resistance) values for astrocyte/hCMEC/D3 direct cocultures compared to astrocyte/hCMEC/D3 indirect cocultures and hCMEC/D3 monocultures. It is interesting to note that astrocytes endocytose rh Bri2 BRICHOS, suggesting that the lower passage of rh Bri2 BRICHOS into the basolateral compartment in the presence of astrocytes, in particular when they grow close to the hCMEC/D3 cells in the direct model, partly is caused by uptake into astrocytes.

### Transcytosis Inhibition

Addition of Dyngo-4a, a dynamin GTPase inhibitor that blocks the formation of endocytotic vesicles (Mccluskey et al., 2013), reduced the passage of rh Bri2 BRICHOS over hCMEC/D3 monolayers more than four-fold. We observed that the presence of Dyngo-4a also led to higher degree of rh Bri2 BRICHOS being taken up or associated with the hCMEC/D3 cells, and that “residual” amounts of rh Bri2 BRICHOS after addition to hCMEC/D3 monolayers, i.e. the amount that could not be accounted for in the apical or basolateral compartments, increased. Notably, the cellular uptake and residual amount of rh Bri2 BRICHOS after 24 h in the presence of Dyngo-4a are both about 5%, which supports that both these measures represent cell-associated rh Bri2 BRICHOS. These results suggest that the passage of rh Bri2 BRICHOS over hCMEC/D3 monolayers requires functional endocytosis, and that blocking the dynamin GTPase activity leads to that rh Bri2 BRICHOS which has bound to hCMEC/D3 cell membranes cannot be endocytosed but accumulates at the cell surface. Unspecific effects of dynamin GTAase inhibitors have been described, and further studies are needed to delineate the molecular details of rh Bri2 BRICHOS transport over hCMEC/D3 monolayers.

To study cellular uptake of rh Bri2 and proSP-C BRICHOS, we used hCMEC/D3 cells, human astrocytes, as well as undifferentiated and differentiated human neuroblastoma derived SH-SY5Y cells, which are widely used as in vitro neuronal models. All BRICHOS variants studied were taken up by the cells, although to different degrees. There is a correlation between passage over hCMEC/D3 monolayers and cellular uptake, where the rh Bri2 BRICHOS monomers show the highest numbers for both parameters. The rh Bri2 BRICHOS oligomers, for which no passage over hCMEC/D3 monolayers could be detected, are taken up to a low degree by undifferentiated and differentiated SH-SY5Y cells, but not by hCMEC/D3 cells. The apparent cellular uptake of rh Bri2 BRICHOS is concentration dependent and shows saturation around 2%, and the residual values determined from the hCMEC/D3 monolayer experiments (i.e. the amounts that cannot be accounted for in the apical or basolateral compartments) are also around 1-2%. The monolayer passage and cell uptake studies, together with the effects observed after dynamin inhibition, strongly support that BRICHOS is transported over the endothelial monolayer by transcytosis, rather than by unspecific leakage. The uptake observed in SH-SY5Y cells and human astrocytes suggest that also neurons and astrocytes can endocytose BRICHOS.

To further rule out the possibility that the BRICHOS passage over hCMEC/D3 monolayers or uptake in hCMEC/D3 and SH-SY5Y cells are related to toxic effects of the proteins studied, we determined cell integrity by propidium iodide (PI) assay and mitochondrial function by tetrazolium-MTT cell assay. The results show that rh Bri2 BRICHOS monomers have no effect on either cell integrity or mitochondrial function, in neither hCMEC/D3 nor SH-SY5Y cells, while rh Bri2 BRICHOS oligomers and proSP-C BRICHOS have minor effects on these cell viability measures. This means that the BRICHOS variants that show highest passage over hCMEC/D3 monolayers are apparently non-toxic, which speaks against the possibility that the passage observed is a result of cytotoxic effects. In addition, TEER values and FITC-dextran permeability measurements of monolayers from day 3-9 do not change in the presence of rh Bri2 BRICHOS, showing that the protein does not have a negative effect on the tight junctions or the overall monolayer integrity.

### Rh Bri2 BRICHOS Fused to Other Proteins is Efficiently Transcytosed Through a Human BBB Model In Vitro and Transported Over the BBB in Wildtype Mice

We used cultured human cerebral microvessel endothelial hCMEC/D3 cell monolayers as an in vitro model of the human BBB (Weksler, B. et al. (2013) Fluids Barriers CNS. 10, 16; and Markoutsa, E. et al. (2011) Eur J Pharm Biopharm. 77, 265-274) to investigate if rh Bri2 BRICHOS can be used as a transport vehicle to facilitate the CNS uptake of other proteins. We tested inter alia the passage of two unrelated proteins fused to rh Bri2 BRICHOS: mCherry, a ca 30 kDa fluorescent protein derived from sea anemones, and NT, a ca 15 kDa protein domain derived from spider silk.

The BRICHOS domain permeability was studied on the hCMEC/D3 cell line, after 6 days of seeding, when the tight junction monolayer had formed with high integrity. Rh Bri2 BRICHOS and proSP-C different constructs were assessed in different concentrations and incubation times and determined by band intensities.

Permeability studies of the recombinant human Bri2 BRICHOS (113-231) wild-type different quaternary structures show that monomers pass the hCMEC/D3 monolayer, but the oligomeric species do not pass. The permeability of the rh Bri2 BRICHOS monomers is time dependent and concentration dependant. Interestingly, Bri2 BRICHOS R221E monomer permeability is higher than that of Bri2 BRICHOS wt monomers. Rh proSP-C BRICHOS wt is determined to cross through the hCMEC/D3 monolayer less than the proSP-C BRICHOS T187R mutant.

TABLE 1
Permeability of different Bri2 and proSP-C BRICHOS domains after
2 h, 24 h or 48 h incubation. 1 μM BRICHOS solutions were
used. Results are represented as a mean of three repeats.

| | | BRICHOS concentration % (Mean ± | | |
|---|---|---|---|---|
| BRICHOS | Time point | SD) | | |
| construct | (h) | Apical | Basolateral | Monolayer |
| Bri2 BRICHOS | 2 | 92.1 ± 1.9 | 6.7 ± 1.4 | 1.2 ± 0.3 |
| monomers | 24 | 86.8 ± 1.8 | 10.4 ± 1.9 | 2.8 ± 0.1 |
| | 48 | 82.4 ± 2.1 | 14.5 ± 2.4 | 3.1 ± 0.3 |
| Bri2 BRICHOS | 24 | 100.0 ± 0.0 | 0.0 ± 0.0 | 0.0 ± 0.0 |
| crude | | | | |
| Bri2 BRICHOS | 24 | 100.0 ± 0.0 | 0.0 ± 0.0 | 0.0 ± 0.0 |
| oligomers | | | | |
| Bri2 BRICHOS | 24 | 80.6 ± 2.2 | 17.7 ± 1.8 | 1.7 ± 0.9 |
| R221E monomers | | | | |
| proSP-C | 24 | 93.7 ± 1.6 | 6 ± 1.8 | 0.3 ± 0.2 |
| BRICHOS wt | 48 | 92.3 ± 2.4 | 7.2 ± 2.5 | 0.5 ± 0.1 |
| proSP-C | 24 | 93.1 ± 3.6 | 6.3 ± 3.7 | 0.6 ± 0.1 |
| BRICHOS T187R | | | | |

TABLE 2
Rh Bri2 BRICHOS wild-type monomer hCMEC/D3 permeability
at different concentrationa and incubation times.
Results are represented as a mean of three repeats.

| Bri2 BRICHOS | | | | |
|---|---|---|---|---|
| monomer | | | | |
| concentration | Time | BRICHOS concentration % (Mean ± SD) | | |
| (μM) | point (h) | Apical | Basolateral | Monolayer |
| 1 | 3 | 90.9 ± 3.3 | 7.8 ± 1.8 | 1.3 ± 1.4 |
| | 24 | 88 ± 2.8 | 10.1 ± 2.3 | 1.9 ± 0.6 |
| 0.5 | 3 | 92.8 ± 3.0 | 5.9 ± 1.3 | 1.3 ± 0.2 |
| | 24 | 89.8 ± 2.0 | 8.5 ± 0.6 | 1.7 ± 1.4 |
| 0.25 | 3 | 97.6 ± 1.8 | 1.2 ± 0.7 | 1.2 ± 1.1 |
| | 24 | 91.8 ± 3.0 | 6.8 ± 3.6 | 1.4 ± 0.6 |
| 0.1 | 3 | 98.6 ± 2.0 | 0.3 ± 0.3 | 1.1 ± 1.1 |
| | 24 | 94.7 ± 3.8 | 4.13 ± 2.3 | 1.2 ± 1.2 |

Permeability of the Bri2 and Bri3 BRICHOS solubility tag conjugates show similarity to unconjugated Bri2 BRICHOS constructs, with the monomeric species crossing the hCMEC/D3 monolayer, but the oligomers or the crude solution not.

NT-Bri2 BRICHOS fusion protein and mCherry-Bri2 BRICHOS fusion protein show the same tendency as the unconjugated Bri2 BRICHOS (113-231) wt and the S-tag fusionprotein, where only the monomers show permeability. On the other hand, the NT-tag separately or the mCherry separately does not show any permeability, demonstrating the Bri2 BRICHOS domain's capability of transporting conjugated molecules through the hCMEC/D3 monolayer.

From the data, it is clear that the endothelial cell uptake and permeability for the BRICHOS domain depends on the quaternary structure, where the monomeric species passes through the hCMEC/D3 monolayer, but the oligomeric does not. The hCMEC/D3 cell line has previously been shown to be size-selective and having restricted permeability in the apical to basolateral direction, with the barrier formation being more efficient towards large molecules. Bri2 BRICHOS R221E monomers and proSP-C BRICHOS T187R monomers show higher permeability due to the formation of stable monomers in comparison to their wild-type counterparts.

TABLE 3
Collective table for 1 μM solutions permeability after 24 h incubation.
Results are represented as a mean of three or four repeats (SD).

| | Concentration % (Mean ± SD) | | |
|---|---|---|---|
| | | Baso- | |
| Molecule of interest | Apical | lateral | Monolayer |
| proSP-C BRICHOS wt | 93.7 ± 1.6 | 6.0 ± 1.8 | 0.3 ± 0.2 |
| proSP-C BRICHOS | 93.1 ± 3.6 | 6.3 ± 3.7 | 0.6 ± 0.1 |
| T187R mutant | | | |
| S-tag-Bri3 BRCHOS | 100.0 ± 0.0 | 0.0 ± 0.0 | 0.0 ± 0.0 |
| (112-230) crude | | | |
| S-tag-Bri2 BRICHOS | 84.9 ± 3.0 | 13.3 ± 2.2 | 1.8 ± 0.5 |
| (90-236) monomers | | | |
| S-tag-Bri2 BRICHOS | 100.0 ± 0.0 | 0.0 ± 0.0 | 0.0 ± 0.0 |
| (90-236) oligomers | | | |
| Bri2 BRICHOS | 86.8 ± 1.8 | 10.4 ± 1.9 | 2.8 ± 0.1 |
| (113-231) wt monomers | | | |
| Bri2 BRICHOS | 100.0 ± 0.0 | 0.0 ± 0.0 | 0.0 ± 0.0 |
| (113-231) wt crude | | | |
| Bri2 BRICHOS | 100.0 ± 0.0 | 0.0 ± 0.0 | 0.0 ± 0.0 |
| (113-231) wt oligomers | | | |
| Bri2 BRICHOS R221E | 80.6 ± 2.2 | 17.7 ± 1.8 | 1.7 ± 0.9 |
| mutant monomers | | | |
| NT-Bri2 BRICHOS | 78.3 ± 1.7 | 20.0 ± 1.6 | 1.7 ± 0.3 |
| monomers | | | |
| NT-Bri2 BRICHOS | 99.3 ± 0.5 | 0.0 ± 0.0 | 0.7 ± 0.5 |
| oligomers | | | |
| mCherry-Bri2 BRICHOS | 82.6 ± 2.8 | 7.3 ± 1.2 | 10.1 ± 1.6 |
| monomers | | | |
| NT*-tag | 98.6 ± 2.0 | 0.0 ± 0.0 | 1.0 ± 1.0 |
| mCherry | 97.0 ± 2.2 | 1.3 ± 1.2 | 1.8 ± 1.7 |
| Human insulin | 85.1 | 13.4 | 1.6 |
| Human apolipoprotein | 94.8 ± 1.4 | 3.7 ± 2.3 | 1.5 ± 0.6 |
| A-I | | | |
| Aβ40 ** | 81.5 ± 4.0 | 0.0 ± 0.0 | 18.5 ± 4.0 *** |
| Aβ42 ** | 73.3 ± 6.1 | 0.0 ± 0.0 | 26.7 ± 6.1 *** |
| FITC-dextran | 98.8 ± 1.2 | 1.2 ± 1.2 | 0.0 ± 0.0 |

** represents a 0.5 μM solution.
*** represents aggregation in the solution.

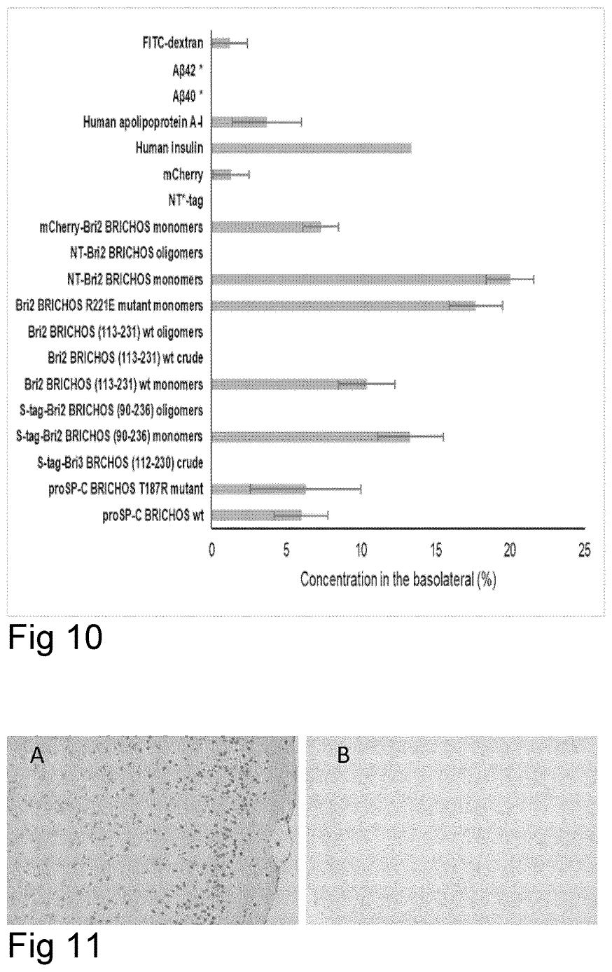

FIG. 10 and Table 3 show hCMEC/D3 monolayer permeability for 1 μM solutions of different proteins and macromolecules (in percent of total amount added to the apical side).

Both rh wildtype and R221E Bri2 BRICHOS monomers show significant passage from the apical side of the monolayers to the basolateral side, i.e. they are transcytosed, while rh wildtype Bri2 BRICHOS oligomers are not (FIG. 10). Neither mCherry nor NT alone passes through the monolayer to any substantial extent, but the fusion constructs with rh Bri2 BRICHOS domain were transcytosed in substantial quantities (FIG. 10).

Whether the substantial passage of Bri2 BRICHOS-mCherry over the human BBB model in vitro can be further increased by addition of microbubbles remains to be determined. And to what extent the human BBB model in vitro predicts all features of the mouse BBB in vivo remains to be determined.

To test whether rh Bri2 BRICHOS works as transport vehicle over the BBB in vivo, we repeated the same type of experiments as previously used to test the BBB passage of recombinant Bri2 and proSP-C BRICHOS in wildtype mice. FIG. 11 shows staining with anti-NT antibody (blue color) of brain cortex of mice injected with NT-Bri2 BRICHOS (A) or PBS (B). This showed that recombinant NT-Bri2 BRICHOS could be detected in brain parenchyma and in neuron cells two hours after intravenous injection, while PBS injected mice showed only background staining (FIG. 11).

These findings strongly support that the ability of rh Bri2 BRICHOS to pass the BBB can be harnessed to transport other proteins over the BBB, and that after passage into the CNS the fusion proteins spread in the parenchyma and are taken up into cells. Proteins generally do not pass the BBB passively, e.g. maximally 0.1% of peripherally administered antibodies pass the BBB (18), which indicates that an active mechanism mediates BBB transfer of rh Bri2 BRICHOS.

NT*-Bri2 BRICHOS monomers passed efficiently also over astrocyte/hCMEC/D3 coculture models. Like for rh Bri2 BRICHOS wt monomers, the passage of the fusion protein over the coculture models, particularly over the astrocyte/hCMEC/D3 direct coculture model, was lower than over the hCMEC/D3 monocultures. Fluorescence images of hCMEC/D3 monolayers after addition of rh Bri2 BRICHOS-mCherry show abundant red fluorescent puncta, while after addition of mCherry alone no such dots are found. Like for rh Bri2 BRICHOS alone, oligomeric forms of the fusion proteins S-tag-Bri2 BRICHOS or NT*-Bri2 BRICHOS do not pass hCMEC/D3 monolayers.

### Rh Bri2 BRICHOS Fused to a Target Protein is Endocytosed and Transported to Lysosomes in Primary Neurons.

The data in FIG. 11A indicate that the fusion protein rh NT-Bri2 BRICHOS is internalized in CNS cells and appears in intracellular vesicles. We studied the intracellular localization in more detail, using primary mouse neurons in culture and the recombinant fusion protein mCherry-Bri2 BRICHOS. Recombinant mCherry-Bri2 BRICHOS was added to the culture medium at low nanomolar concentrations, and after 24-48 h the neurons were immobilized and visualized under a fluorescence microscope.

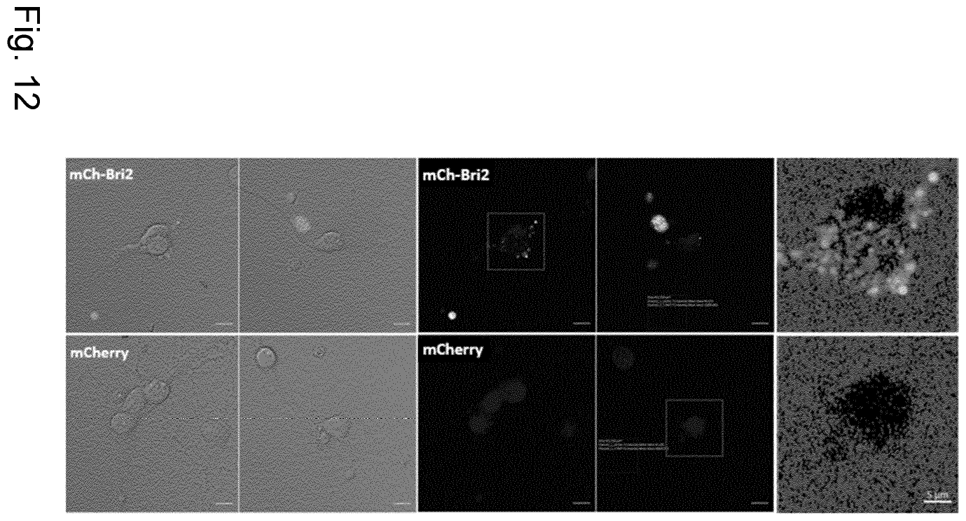

FIG. 12 shows that Rh Bri2 BRICHOS mediates uptake into mouse primary neurons. A fusion protein consisting of Bri2 BRICHOS linked to mCherry (denoted mCh-Bri2, results in upper row) is detected in abundant intracellular vesicles after addition to the culture medium. mCherry alone (results in lower row) is not taken up.

Thus, mCherry-Bri2 BRICHOS is endocytosed and can be detected in intracellular vesicles, while the control protein mCherry alone is not taken up to any detectable level. These data are in excellent agreement with the results from the transcytosis experiments in FIG. 10.

Finally, Rh Bri2 BRICHOS linked to a cargo protein is endocytosed and transported to lysosomes. Primary mouse neurons which were treated with the fusion protein mCherry-Bri2 BRICHOS show intracellular uptake and presence in lysosomes as shown by co-localization with the lysosome-specific dye SiR-Lyso (data not shown). Thus, labeling of the neurons that had been treated with mCherry-Bri2 BRICHOS with the lysosome-specific dye SiR-lysosome showed clear co-localization.

### CONCLUSIONS

The present study shows that rh Bri2 BRICHOS is efficiently transported over hCMEC/D3 monolayers, an in vitro model of the human BBB, apparently via a dynamin-dependent endocytosis mechanism. The BRICHOS mechanisms of action in vivo are apparently similar to those found to apply in vitro and ex vivo, suggesting that selective reduction of toxic oligomers by Bri2 BRICHOS is an important part of the treatment effects. The results shown herein that rh Bri2 BRICHOS can enter neurons and astrocytes after intravenous injection in mice suggest that Bri2 BRICHOS can mediate cellular effects as well. Bri2 BRICHOS can carry cargo proteins over the human BBB models used here, and this property apply to the BBB in vivo as well, as rh NT*-Bri2 BRICHOS can be found in early endosomes in neurons and in astrocytes in major parts of the brain after intravenous injection in mice. This opens the exciting possibility that BRICHOS capacity to reduce Alzheimer pathology can be coupled to additional activities mediated by fusion partners, thereby potentially generating multimodal biologic drugs that also efficiently pass the BBB. One candidate group for fusion with Bri2 BRICHOS are monoclonal antibodies against Aβ, which have shown therapeutic efficiency in clinical trials for Alzheimer's disease (van Dyck et al., N Engl J Med 388 (1): 9-21, 2023), but likely would benefit from increased BBB passage and from coupling them to the effects against Aβ toxicity mediated by Bri2 BRICHOS. Notably, BRICHOS is more potent in reducing the number of toxic Aβ oligomers in vitro compared to monoclonal antibodies against Aβ that have undergone clinical trials (Linse et al., Nat Struct Mol Biol, 27:1125-1133, 2020). Bri2 BRICHOS combines rather small size, scalable recombinant production, lack of detectable toxicity to cells in culture or after repeated intravenous injections, and high capacity to carry cargo over the BBB. Preparation and administration protocols for rh Bri2 BRICHOS fusions are also rather straightforward compared to making transport vesicles via encapsulation strategies. This makes rh Bri2 BRICHOS an interesting vehicle for delivery of various macromolecules over the BBB and into cells in the CNS.

### Experiments Regarding Inhibition of α-Synuclein Oligomer Generation

### Materials and Methods

### Preparation of Bri2 BRICHOS Monomers

SHuffle T7 (K12 strain) E. coli cells were transformed with a plasmid vector carrying the human Bri2 BRICHOS R221E mutant sequence (113-231 of full-length human Bri2) fused to His6x-NT at the N-terminus separated by a thrombin cleavage site. The cells were incubated at 30 °C in LB medium containing 15 μg·ml⁻¹ kanamycin until reaching OD=0.8. The cells were then induced with 0.5 mM IPTG and grown overnight at 20 °C. The cells were harvested at 5000×g centrifugation at 4 °C and pellets were resuspended into 20 mM Tris HCl, pH 8.0. Two pellets were sonicated for 5 min (2 s on, 2 s off, 65% of max power) and the resulting lysate centrifuged at 20000×g, 4° C. for 30 min. The collected supernatant was then loaded on a Ni-NTA column (GE healthcare Bio-Science Aβ, Sweden) The column was subsequently washed with 30 column volumes (CV) with 20 mM Tris HCl, 15 mM imidazole, pH 8.0 and followed with elution using 20 mM Tris HCl, 300 mM imidazole, pH 8.0. Fractions were pooled and dialyzed against 20 mM Sodium Phosphate, 0.2 mM EDTA, pH 7.4 at 4 °C. O/N with an addition of thrombin (1:1000 enzyme to substrate, w/w). The sample was then loaded on Ni-NTA gravity columns to remove His-NT species and uncleaved proteins of interest. The flowthrough was concentrated using 10 kDa cut off concentrator (Vivaspin 20, 10 kDa MWCO Cytiva) and directly loaded on Hiload superdex 75 PG Size Exclusion column (Cytiva, USA) to collect the peak of interest corresponding to the monomer.

### Preparation of α-Syn Monomers

BL21 (DE3) E. coli were transformed with the bacterial expression plasmid pET21α-alpha-synuclein (Addgene) containing the human α-syn coding sequence. The cells were grown at 37 °C in LB medium containing 100 ug·ml-1 Ampicillin until reaching OD=0.8. The culture was inoculated with 0.5 mM IPTG and incubated at 20 °C O/N. The cells were centrifuged at 5000×g for 20 min at 4 °C and the bacterial pellets were resuspended by gentle vortexing in 20 mM Tris-HCl, pH 8.0. A cell pellet from 1 L was warmed up and lysed using a sonicator (G. Heinemann Ultraschall-un Labortechnik) at 65% maximum power for 5 min with 2 sec ON, 2 sec OFF pulse setting. The sample was then boiled for 10 or 5 min. This was followed by centrifugation at 24000×g for 20 min at 4 °C. The supernatant was loaded onto a HiTrap QFF anion exchange chromatography column. The protein was eluted with a gradient of 20 mM Tris HCl, 1 M NaCl, pH 8 during 10 CV. The eluted peak was then loaded on a reverse phase chromatography column using 99.9% H₂O, 0.1% TFA as the running buffer. Elution was done using 70% ACN, 0.1% TFA buffer gradient during 20 CV. The fractions corresponding to the peak of interest were pooled and the optical density at 280 nm was measured. The samples were lyophilised O/N using a vacuum (LabConco CentriVap Concentrator). The lyophilized samples that were not used directly were stored at −20 °C. For further purification, around 8 mg of lyophilised protein aliquots were dissolved in 550 μl 7 M Gdn-HCl, pH 8.0 and loaded onto the Superose 6 Increase column with 20 mM Sodium phosphate, 0.2 mM EDTA, pH 7.4 as running buffer in order to obtain homogenous and pure α-syn monomer sample further stored at −20 °C.

### ThT Aggregation Assays

For kinetics experiments, each sample contained either 30 or 70 μM of α-syn monomers in the presence of 20 μM ThT and different concentration of Bri2 BRICHOS monomers in 20 mM sodium phosphate, 0.2 mM EDTA, pH 7.4 buffer or with added NaCl salts at 154 mM. Two glass beads of 1 mm diameter were used in each well. The fluorescence was recorded using a 448-10 nm excitation filter and a 482-10 nm emission filter (FLUOStar Omega from BMG Labtech, Offenberg, Germany) while the plate was orbitally shaken between cycles at 300 rpm for 9 min ON and 1 min OFF. Each cycle was set to 10 min long and the temperature was set to 37 °C. For preparation of seeds, α-syn fibrils were obtained from previous runs of ThT kinetics using 70 μM α-syn monomers alone. These fibrils were then sonicated during 3 min, 2 sec ON, 2 sec OFF using a probe sonicator (SONICS Vibra Cell™). For all experiments, aggregation traces were normalized and averaged using 6 replicates for each sample.

### Analysis of α-Syn Aggregation Kinetics

Aggregation traces of 70 and 30 μM α-syn monomers with different concentrations of BRICHOS in molar ratio between 0 and 100% were normalized, averaged over 6 replicates and truncated at the plateau of the aggregation reaction. The aggregation kinetics were fitted by applying the secondary nucleation dominated, unseeded nucleation model in Amylofit which includes primary, secondary nucleation and elongation as combined rates k+kₙ and k+k₂. An individual fit was performed first to obtain the dependence of these parameters on the BRICHOS concentration. To evaluate the contribution of a single nucleation rate, we then applied the secondary nucleation dominated with initial aggregate number and concentration set at 0. Two nucleation rate constants are set as global fit parameters, meaning that they are constrained to the same value across all BRICHOS concentrations, while one nucleation constant was set as an individual fitting parameter.

Highly seeded aggregation kinetics were analysed using GraphPad. Elongation rates were extracted by fitting a straight line to the first 2 h of the aggregation curves.

Preparation of Fibrils Incubated with Bri2 BRICHOS

α-syn monomers (70 μM) were incubated similarly as for the ThT kinetic experiment but without ThT. After reaching around 100 h of experiment, the fibrils were spun down at 16200×g at 4 °C for 30 min and washed twice with fresh buffer (20 mM sodium phosphate buffer 0.2 mM EDTA pH 7.4).

### Transmission Electron Microscopy

5 μl diluted solution of α-syn fibrils (around 15 μM) was applied on 200 mesh formvar coated nickel grid and excess solution was removed using blotting paper after 10 min of incubation. It was then washed twice with 10 μl MQ water and subsequently stained with 1% uranyl formate for 5 min. Extra stained was blotted with blotting paper and it was air-dried. Transmission electron microscopy (FEI Tecnai 12 Spirit BioTWIN, operated at 100 kV) was performed for analysis of fibril morphology using 2 k×2 k Veleta CCD camera (Olympus Soft Imaging Solutions, GmbH, Münster, Germany). 15-20 images were recorded for each sample randomly and fibril diameter was measured using Image J software.

### Native PAGE

BRICHOS was added at equimolar concentration to α-syn fibrils sample (8 μM). Samples were prepared under non-denaturing conditions and subsequently run using Native gel at 7.5%. BRICHOS was visualized using Coomassie blue staining.

### Oligomers Capture

α-syn monomers (70 μM) with and without equimolar concentration of BRICHOS were incubated similarly as for the ThT kinetic experiment. Samples were collected at different time points and snap-freezed in liquid nitrogen and kept at −80 °C until further use. The samples were prepared under non-denaturing conditions, and run on Native PAGE 10% and Coomassie blue stained.

### Surface Plasmon Resonance

The binding analysis was performed using a Biacore 3000 instrument at 25 °C. Different ChiP were used to perform these experiments: α-syn fibrils, salted α-syn fibrils and α-syn monomers which have been previously immobilized.

The system was primed with the following running buffer: 20 mM sodium phosphate, 0.2 mM EDTA, pH 7.4. The different concentration of Bri2 BRICHOS (0, 1.56, 3.125, 6.25, 12.5, 25, 50, 100 μM) were injected for 5 minutes at flow rate 20 μl/min over the channels 2 and 4. Channels 1 and 3 were left empty and served as a reference to control for possible nonspecific binding of α-syn. BRICHOS proteins, if bound, were washed away with 20 mM NaOH and buffer was equilibrating the chip for 15 minutes before injecting the sample again. Each sample was run in duplicates.

### NMR Experiments

¹H-¹⁵N HSQC titration were conducted with 15N-labeled α-syn or 15N-labeled BRICHOS in 20 mM NaP, 0.2 mM EDTA at pH 7.4 with addition of either BRICHOS or α-syn sonicated fibrils respectively at 281 K on a Bruker 700 Mhz spectrometer. All NMR data were processed using Bruker Topspin and analysed with Poky.

### ITC

216 μM BRICHOS monomers was injected 18 times with the injection syringe of 2 μl in the sample cell containing 20 μM α-syn monomers or buffer (20 mM NaP, 0.2 mM EDTA, pH 7.4) for the control (iTC200 α-syn Microcal, GE Healthcare). The experiment was performed at room temperature and stirring speed at 1000 rpm.

### FIDA

Recombinant wild-type human α-syn was expressed in E. coli using a pET11-D construct and purified as previously described (Lorenzen et al., J. Am. Chem. Soc., 136:3859, 2014; Paslawski et al., Methods Mol. Biol., 1345:133, 2016). Oligomeric and monomeric α-syn were prepared by 3 h incubation of 8 mg/ml pure α-syn dissolved in PBS at 37 °C with 900 rpm shaking on a Heating Digital Shaking Drybath (Thermo Scientific), followed by size exclusion on a Superose 6 prep grade XK 26/100 column (GE Healthcare). Monomer and oligomer fractions were collected at stored at 4 °C. Prior to analysis, the oligomer fractions were pooled and concentrated using a 100 kDa spin filter (Amicon Ultra). α-syn fibrils were prepared by dissolving freezedried α-syn in PBS and setting the filtered solution to fibrillate for 3 days at 37 °C at 1.1 mg/mL concentration while shaking at 600 rpm in a 96-well plate with a 3 mm glass bead. The fibrils were separated by centrifugation at 13.000 rpm for 10 min, redissolved in PBS to 1 mg/ml concentration, and fragmented by sonication using a Q500 sonicator (Qsonica, Connecticut USA) mounted with 2 mm Microtip probe, set to 10-s pulses with 10-s pause at 20% intensity for a total 30-s sonication. BRICHOS was labelled with Alexa-488 NHS Ester (Thermo Scientific) by mixing 43.6 μM BRICHOS dissolved 100 mM bicarbonate buffer (pH 8.3) and Alexa-488 dissolved in DMSO in a 1:2 molar ratio followed by 1 hour incubation at room temperature. The reaction was quenched by addition of 100 mM Tris-HCl pH 7.4 and the labelled BRICHOS was separated from unreacted label by a PD-10 column (GE Healthcare) equilibrated with PBS. Protein concentration and labeling efficiency were estimated using DeNovix DS-11 (DeNovix). Flow-induced dispersion analysis was performed on a FIDA One instrument (FIDA Biosystems) using LED-induced fluorescence detection with excitation wavelength at 480 nm. Standard capillary (inner diameter: 75 μm, outer diameter: 375 μm, length total: 100 cm, length to detection window: 84 cm) (Fida Biosystems) was rinsed by 1 M NaOH and equilibrated with PBS at 25 °C. The experiments were performed by priming the capillary by running buffer at a pressure of 3500 mbar. Subsequently, the analyte (α-syn monomer, oligomer, or fibril) at various concentrations and indicator (BRICHOS-Alexa488) at 50 nM were sequentially injected into the capillary for 20 seconds each, with a pressure of 3500 mbar for the analyte injection and 50 mbar for the indicator injection, respectively. To enable the movement of the indicator and analyte towards the detector, a final injection of the analyte was performed for 180 seconds at a pressure of 400 mbar. The resulting Taylograms were analyzed using Fida software V2.3 (FIDA Biosystems), which provides hydrodynamic radius (Rₕ) for each binding reaction. The resulting isothermal binding curves were fit to the following equation:

$${R}_{app} = \frac{1 + \frac{1}{{K}_{D}} [ A ]}{( {( {R}_{I} )}^{- 1} - {( {R}_{IA} )}^{- 1} ) + ( 1 + \frac{1}{{K}_{D}} [ A ] ) {( {R}_{IA} )}^{- 1}} \qquad 1$$

where K_{D} is the dissociation constant, [A] is the analyte concentration, and R_{I} and R_{IA} are the Rₕ of the indicator and the complex, respectively.

### Electrophysiological Studies

For electrophysiological experiments, all chemical compounds used in extracellular solutions were obtained from Sigma-Aldrich Sweden Aβ (Stockholm, Sweden). Kainic acid (KA) was obtained from Tocris Bioscience (Bristol, UK). α-syn fibrils were prepared by incubating α-syn monomers at 37 °C at 900 rpm in PBS, 0.1% Sodium Azide, pH 7.4 for 7 days. They were then sonicated during 3 min, 2 sec ON, 2 sec OFF using a probe sonicator.

We used wild-type (WT) mice (n=12) at 4-6 postnatal weeks to test the effect of in vitro α-syn fibrils by incubating on ex vivo hippocampal gamma oscillations. For brain extraction, mice were deeply anesthetized with isoflurane. The brain was dissected out and placed in ice-cold artificial cerebrospinal fluid (ACSF) modified for dissection containing (in mM): 80 NaCl, 24 NaHCO₃, 25 glucose, 1.25 NaH₂PO₄, 1 ascorbic acid, 3 Na-pyruvate, 2.5 KCl, 4 MgCl₂, 0.5 CaCl₂), 75 sucrose and bubbled with carbogen (95% O₂ and 5% CO₂). Horizontal sections (350 μm thick) of the ventral hippocampi of both hemispheres were prepared with a Leica VT1200S vibratome (Leica Microsystems). Immediately after cutting, slices were transferred into a humidified interface holding chamber containing standard ACSF (in mM): 124 NaCl, 30 NaHCO₃, 10 glucose, 1.25 NaH₂PO₄, 3.5 KCl, 1.5 MgCl₂, 1.5 CaCl₂), continuously supplied with humidified carbogen. The chamber was held at 37 °C during slicing and subsequently allowed to cool down to room temperature (~22 °C) for a minimum of 1 hour. We selected four conditions to test the toxic effect of α-syn and BRICHOS R221E monomers on hippocampal gamma oscillations: Sham group (PBS, 0.1% Sodium Azide, pH 7.4), α-syn 500 nM, α-syn 1 μM and α-syn 1 μM+Brichos 1 μM. We preincubated hippocampal slices in each condition for 30 min in a submerged incubation chamber containing ACSF. During the incubation, slices were supplied continuously with carbogen gas (5% CO₂, 95% O₂) bubbled into the ACSF. After incubation time the slices were transferred to the interface-style recording chamber for extracellular recordings.

Recordings were performed with borosilicate glass microelectrodes filled with ACSF in hippocampal area CA3, pulled to a resistance of 3-6 MΩ. Local field potentials (LFP) were recorded at 32 °C in an interface-type chamber (perfusion rate 4.5 mL per minute). LFP gamma oscillations were elicited by kainic acid (100 nM). The oscillations were stabilized for 20 min before any recordings. Interface chamber LFP recordings were carried out by a 4-channel amplifier/signal conditioner M102 amplifier (Electronics lab, University of Cologne, Germany). The signals were sampled at 10 KHz, conditioned using a Hum Bug 50 Hz noise eliminator (Quest Scientific, North Vancouver, BC, Canada), software low-pass filtered at 1 KHz, digitized, and stored using a Digidata 1322 A and Clampex 10.4 programs (Molecular Devices, CA, USA). Power spectra density plots (from 60 s long LFP recordings) were calculated in averaged Fourier-segments of 8192 points using Axograph X (Kagi, Berkeley, CA, USA). Gamma oscillations power was calculated by integrating the power spectral density between 20 and 80 Hz with the result representing average values taken over 1 min periods.

### Results

### BRICHOS Efficiently Retards α-Syn Bulk Aggregation

We first followed the aggregation kinetics of 70 μM α-syn using shaking conditions at physiological pH 7.4 in the presence of different concentration of recombinant human (rh) Bri2 BRICHOS R221E monomers (referred to as BRICHOS below) ranging from 2 to 95% molar equivalent of monomeric α-syn. We observed that the aggregation of α-syn was delayed upon addition of BRICHOS in a concentration dependent manner up to 95% molar equivalent of BRICHOS. This results in a delay of the aggregation half time from 18.3±1.8 h to 53.1±3 h, obtained by sigmoidal fits to the individual aggregation traces. At higher BRICHOS concentration the inhibitory effect appears to be saturated and no further retardation of the aggregation is observable starting from 25% to 100% BRICHOS.

To test whether the inhibitory effect can be shielded by electrostatic interactions, we conducted the same experiment in the presence of physiological salt concentration. We found that the presence of salt greatly accelerates the aggregation kinetics of α-syn with an aggregation half time of 9.6±0.5 h compared to 18.3±1.8 without salt. Notably, also under these conditions BRICHOS efficiently retards the aggregation process, delaying the aggregation half time to 12.1±5 h at 90% molar equivalent. Hence, the inhibitory effect of BRICHOS is specific to α-syn and works in near-physiological conditions.

We further aimed to confirm our results by performing the same experiments at lower α-syn concentration, using 30 instead of 70 μM, and adding the same molecular ratios of BRICHOS. We indeed found that BRICHOS also delays α-syn aggregation at lower α-syn concentration both with and without the presence of salt. These results indicate that inhibitory effect is specific of BRICHOS interacting with strong electrostatic forces or other type of interactions such as hydrophobic interactions with α-syn.

### BRICHOS Specifically Inhibits Secondary Nucleation and Fibril-End Elongation

Amyloid formation can generally be described by a nucleation-dependent polymerization mechanism. The formation of a nucleus, known as primary nucleation (kₙ), triggers the growth of additional fibrils through fibril-end elongation (k₊). Other secondary processes can also occur such as secondary nucleation (k₂) or fragmentation (k₋). α-syn aggregation traces can be fitted with these theoretical kinetics models, describing the contributions of specific microscopic nucleation events. Here, we applied a model consisting of three microscopic processes: primary nucleation, secondary nucleation and fibril-end elongation elongation. In addition to monomer-dependent secondary nucleation, monomer-independent secondary reactions might be present, which are related to fibril fragmentation, and described by the nucleation rate constant k-. Typically, this process can be related to agitation during the fibril reaction.

Previous studies have shown that α-syn aggregation, follows a secondary nucleation dependent aggregation mechanism at physiological pH. Conducting a detailed global fit analysis of the aggregation kinetics we could analyse which nucleation rates are affected by BRICHOS. First, we applied a model that only includes monomer-dependent secondary nucleation reactions, in addition to primary nucleation and elongation.

To decipher the contribution of the individual nucleation events, we set two nucleation rate constants as global fit parameters, meaning that they are constrained to the same value across all BRICHOS concentrations, and one nucleation constant was set as an individual fitting parameter. The global fits that described best the data were the one where either k₂ or k₊ were free fitting parameters, suggesting that BRICHOS suppresses elongation and/or secondary nucleation. The same results were obtained in the presence of salts and at 30 μM α-syn in the presence of salt.

Second, we tested a different nucleation model where monomer-dependent secondary nucleation, k₂, is neglected and replaced by fragmentation-related monomer-independent secondary nucleation, k₋. Also for this model we obtained overall good fits for either k₋ or k₊ as the free fitting parameters, yet the mean residual errors slightly favour the monomer-dependent secondary nucleation model.

To deduce whether BRICHOS affects predominately elongation or secondary nucleation, or both, we performed highly seeded kinetics. At high seed concentration a large number of free fibril ends is available, creating a condition where the start of the aggregation kinetics is dominated by only fibril elongation events. Hence, by analysing the initial slope of the first 2 h of the kinetic traces, we could extract the elongation rates. We found that the initial slope is dependent on the BRICHOS concentration, indicating that BRICHOS prevents the elongation of α-syn fibrils. Comparing the dependence the combined fitting parameter k₊k₂ from the global fit analysis with the initial slope, which proportional to k₊, from the highly seeded aggregation kinetics, we found that the modulation in k₊ cannot explain only the large effect observed on k₊k₂. This suggests that k₂ is also largely reduced by the presence of BRICHOS, in addition to k₊. Hence, these results show that BRICHOS suppresses both secondary nucleation and fibril-end elongation.

While fragmentation-related processes might be present as well, a model including both k₋ and k₂ exhibits several coupled fitting parameters, which increases the risk for over-fitting. The slightly better fits for k₂, together with that the inhibition of secondary nucleation on the fibrils surface can be mechanistically understood by bound BRICHOS on α-syn fibrils, provide a reductionist explanation about the mechanism-of-action where BRICHOS prevents monomer-dependent secondary nucleation and fibril-end elongation.

### BRICHOS has a Decreased Inhibitory Effect on the Aggregation of C-Terminally Truncated α-Syn Variants

Subsequently, we investigated the effect of BRICHOS on C-terminally truncated variants of α-syn at positions 110 and 121, which are referred to as α-syn110 and α-syn121, respectively. These variants have been shown to exhibit an accelerated propensity for aggregation (Farzadfard et al., Commun Biol, 5 (1): 123, 2022). Our findings revealed that BRICHOS has only a limited effect on the α-syn121 and small inhibitory effect on the α-syn110, whose data cannot be fitted. These results suggest that the presence of the C-terminus is essential for optimal efficacy of BRICHOS to prevent α-syn aggregation. Given that secondary nucleation involves interaction between the C-terminal segment of α-syn fibrils and the N-terminus of α-syn monomers (Kumari et al., Proc Natl Acad Sci USA, 118 (10), 2021), the limited effect of BRICHOS on aggregation kinetics of C-terminally truncated variants suggests that BRICHOS binds to the C-terminus of α-syn competiting with α-syn monomers for secondary nucleation processes.

BRICHOS does not Affect the Morphology of α-Syn Fibrils

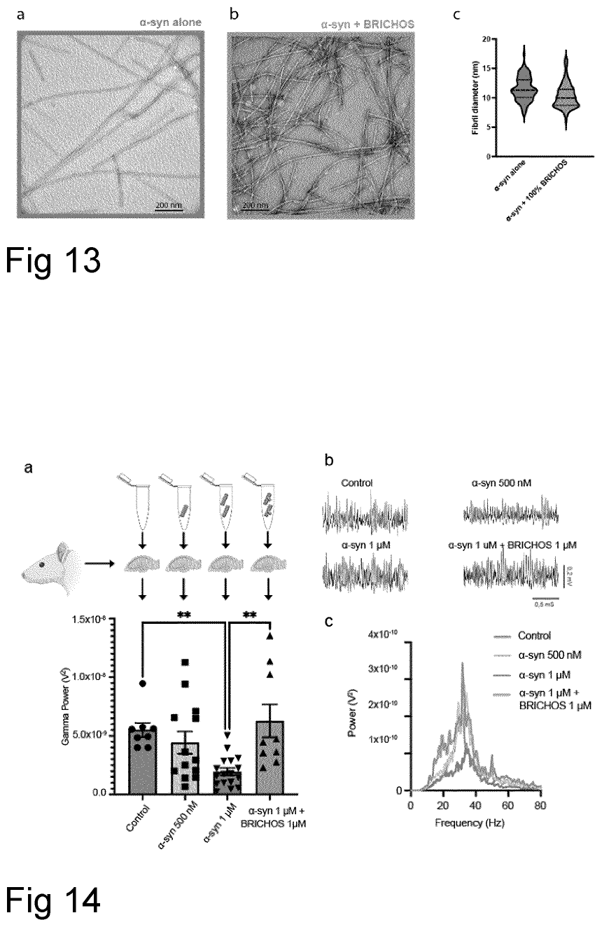

To investigate whether BRICHOS modulates the α-syn fibril morphology, we collected electron microscopy images of α-syn fibrils, obtained without and with 100% molar equivalent of BRICHOS during the aggregation reaction. We analysed the diameter of 100 selected fibrils for both conditions, and observed that the diameters of the BRICHOS co-incubated α-syn fibrils of the first generation were about 10.32±2.07 nm and 11.52±1.10 nm for α-syn fibrils alone (FIG. 13) which is in agreement with published in vitro α-syn fibrils 3D structure which are around 10 nm corresponding to two protofilaments bundled together.

FIG. 13 shows that BRICHOS does not affect α-syn fibril morphology. TEM images of α-syn fibrils after fibrillization at 70 μM without (a) or with 100% (b) BRICHOS molar equivalent. (c) Fibril diameter of α-syn fibril incubated without or with BRICHOS.

In conclusion, BRICHOS does not influence the diameter of α-syn fibrils when co-incubated with α-syn monomers.

### BRICHOS Binds to α-Syn Fibrils but not Monomers

To assess the binding capacity of BRICHOS with different α-syn species, we employed various biophysical techniques, including Isothermal Titration calorimetry (ITC), Surface Plasmon Resonance (SPR) and Fluorescence Intensity Distribution Analysis (FIDA). SPR experiments confirmed the binding of BRICHOS to α-syn fibrils.

To determine the amount of BRICHOS bound to α-syn fibrils, co-incubated BRICHOS-α-syn fibrils samples were analyzed using Native PAGE. These experiments suggested that only 9±4% of BRICHOS molecules are bound to the fibrils when incubated at equimolar concentrations. This observation was confirmed by solution NMR experiments recording 2D ¹H-¹⁵N HSQC spectra, where sonicated α-syn fibrils were added to ¹⁵N-labeled BRICHOS. Due to the dynamic nature of BRICHOS, only parts of the expected resonance are visible in spectrum and bound BRICHOS is expected to be invisible in solution NMR spectra due to large size of α-syn fibrils. We found that addition of α-syn fibrils at equimolar concentration resulted in disappearance of around 10±10% of the signals, indicating that only a small proportion of BRICHOS is bound. Furthermore, the addition of either half or twice the molar concentration of fibrils resulted in a similar loss of signal.

Fluorescence in-capillary FIDA experiments were conducted using fluorescently labelled BRICHOS-Alexa488 to investigate its binding affinities to α-syn fibrils and monomers. While BRICHOS-Alexa488 did not show any binding to α-syn monomers, higher molecular species were detected in the presence of fibrils, indicating binding between BRICHOS-Alexa488. Moreover, NMR HSQC experiments were performed to validate these findings by using 15N labelled α-syn which showed no observable binding between BRICHOS and α-syn monomers. This lack of interaction was further supported by ITC experiments which demonstrated that BRICHOS does not bind to α-syn monomers and signal observed is only due to heat of dilution of the BRICHOS sample being injected in the component cell.

### BRICHOS Binds to α-Syn Oligomers

We further investigated the ability of BRICHOS to bind α-syn oligomers. One challenge that we faced was coming from the complexity of generating α-synuclein oligomers, as various oligomeric forms can emerge under different environmental conditions, exhibiting different characteristics and diverse structural arrangements. In our study, α-synuclein oligomers were prepared by incubating α-synuclein monomers to 37 °C during 5 h, followed by the isolation of the oligomeric species through size exclusion chromatography after the removal of larger aggregates by centrifugation. These particular oligomers are considered stable, show β-sheet content, are kinetically trapped and able to permeabilize vesicles in vitro. Using the FIDA experiment, we observed that BRICHOS-Alexa488 can bind these α-syn oligomers. By conducting a simulation using kinetic parameters derived from experimental data, we can observe that the presence of BRICHOS leads to a reduction in secondary nucleation rate therefore in the formation of oligomers.

### BRICHOS Prevents α-Syn Neurotoxicity

Subsequently, we assessed how the impact of BRICHOS on α-syn aggregation translated into α-syn neurotoxicity. Electrophysiological assays were conducted on hippocampal brain slices from wild-type mice. Characteristics of γ-oscillations are great indicator of neuronal activity and neurotoxicity of added compounds. Following exposure to control buffer (PBS, 0.1% Sodium Azide, pH 7.4), sonicated α-syn fibrils alone or in combination with equimolar concentrations of BRICHOS, γ-oscillations were recorded (FIG. 14, α-c).

FIG. 14 shows that BRICHOS monomers prevents a neurotoxic effect of sonicated α-syn fibrils.
(a) Schematic experimental set up where toxicity is measured on hippocampal mouse brain slices.
(b) Example traces of γ-oscillations under control condition (gray), after incubation with 500 nM α-syn sonicated fibrils, 1 μM of sonicated α-syn fibrils and 1 μM of sonicated α-syn fibrils with equimolar concentration of BRICHOS.
(c) Example power spectra of γ-oscillations under control condition, after incubation with 500 nM α-syn soniciated fibrils, 1 μM of sonicated α-syn fibrils and 1 μM of sonicated α-syn fibrils with equimolar concentration of BRICHOS.

Sonicated α-syn fibrils showed a concentration-dependent impact on the power of γ-oscillations. At 500 nM fibril concentration did not result in any significant reduction in γ-oscillation power, while a concentration of 1 μM led to a substantial decrease in γ-oscillation power, indicating notable neurotoxic effects. Interestingly, co-incubation of 1 μM α-syn sonicated fibrils with BRICHOS at an equimolar concentration did not impact the power of γ-oscillations, suggesting that the presence of BRICHOS effectively prevented the neurotoxic effects of α-syn fibrils.

### ITEMIZED LIST OF EMBODIMENTS

1. An isolated recombinant protein selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4); for use in a method of treatment of Alzheimer's disease in a mammal, including man, in need thereof comprising the steps of;
   administrating to said mammal a plurality of lipid microbubbles and/or nanodroplets
   administrating to said mammal said isolated recombinant protein;
wherein said isolated recombinant protein is not comprised within said microbubbles and/or said nanodroplets; and
wherein the method is not comprising any step of ultrasound treatment of any tissue of the mammal.
2. An isolated recombinant protein selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4); for use in a method of treatment of Alzheimer's disease in a mammal, including man, in need thereof consisting of the steps of;
   administrating to said mammal a plurality of lipid microbubbles and/or nanodroplets
   administrating to said mammal said isolated recombinant protein;
wherein said isolated recombinant protein is not comprised within said microbubbles and/or said nanodroplets.
3. A combination of an isolated recombinant protein and lipid microbubbles and/or nanodroplets according to any one of the preceding items for use in a method of treatment of Alzheimer's disease in a mammal, including man, in need thereof comprising the steps of;
   administrating to said mammal a plurality of lipid microbubbles and/or nanodroplets
   administrating to said mammal said isolated recombinant protein;
wherein said isolated recombinant protein is not comprised within said microbubbles and/or said nanodroplets; and
wherein the method is not comprising any step of ultrasound treatment of any tissue of the mammal.
4. A combination of an isolated recombinant protein and lipid microbubbles and/or nanodroplets according to any one of the preceding items for use in a method of treatment of Alzheimer's disease in a mammal, including man, in need thereof consisting of the steps of;
   administrating to said mammal a plurality of lipid microbubbles and/or nanodroplets
   administrating to said mammal said isolated recombinant protein;
wherein said isolated recombinant protein is not comprised within said microbubbles and/or said nanodroplets.
5. An isolated recombinant protein or combination for use according to any one of the preceding items, wherein said isolated recombinant protein and said microbubbles and/or said nanodroplets are administered intravenously.
6. An isolated recombinant protein or combination for use according to any one of items 1-5, wherein a therapeutically effective amount of at least 1 mg/kg, such as at least 5 mg/kg, more preferably such as at least 10 mg/kg an of said isolated recombinant protein is administered.
7. An isolated recombinant protein or combination for use according to any one of items 1-5, wherein a therapeutically effective amount of less than 50 mg/kg, such as less than 30 mg/kg, more preferably less than 20 mg/kg of said isolated recombinant protein is administered.
8. An isolated recombinant protein or combination for use according to any one of the items 1-7, wherein the isolated recombinant protein is selected from the group consisting of proteins comprising an amino acid sequence having at least 70%, preferably at least 75%, identity to residues 113-231 of Bri2 from human (SEQ ID NO: 2); and proteins comprising an amino acid sequence having at least 70%, preferably at least 75%, identity to the BRICHOS domain of Bri2 from human (SEQ ID NO: 5).
9. An isolated recombinant protein or combination for use according to item 8, wherein the isolated recombinant protein is selected from the group consisting of proteins comprising an amino acid sequence having at least 80%, preferably at least 85%, identity to residues 113-231 of Bri2 from human (SEQ ID NO: 2); and proteins comprising an amino acid sequence having at least 80%, preferably at least 85%, identity to the BRICHOS domain of Bri2 from human (SEQ ID NO: 5).
10. An isolated recombinant protein or combination for use according to item 9, wherein the isolated recombinant protein is selected from the group consisting of proteins comprising an amino acid sequence having at least 90%, preferably at least 95%, preferably at least 99%, identity to residues 113-231 of Bri2 from human (SEQ ID NO: 2); and proteins comprising an amino acid sequence having at least 90% identity to the BRICHOS domain of Bri2 from human (SEQ ID NO: 5).
11. An isolated recombinant protein or combination for use according to item 10, wherein the isolated recombinant protein is selected from the group consisting of residues 113-231 of Bri2 from human (SEQ ID NO: 2); and the BRICHOS domain of Bri2 from human (SEQ ID NO: 5).
12. An isolated recombinant protein or combination for use according to any one of items 1-7, wherein the isolated recombinant protein is selected from the group of proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
13. An isolated recombinant protein or combination for use according to item 12, wherein the isolated recombinant protein is selected from the group of proteins comprising an amino acid sequence having at least 80%, preferably at least 85%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
14. An isolated recombinant protein or combination for use according to item 13, wherein the isolated recombinant protein is selected from the group of proteins comprising an amino acid sequence having at least 90%, preferably at least 95%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
15. An isolated recombinant protein or combination for use according to item 14, wherein the isolated recombinant protein is selected from the group consisting of any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
16. An isolated recombinant protein or combination for use according to any one of the items 1-15, wherein, in the isolated recombinant protein, the amino acid residue corresponding to position 221 in SEQ ID NO: 1 is selected from the group consisting of Glu and Asp.
17. An isolated recombinant protein or combination for use according to item 16, wherein, in the isolated recombinant protein, the amino acid residue corresponding to position 221 in SEQ ID NO: 1 is Glu.
18. An isolated recombinant protein or combination for use according to any one of the items 1-17 wherein the isolated recombinant protein is consisting of less than or equal to 200 amino acid residues, such as less than or equal to 150 amino acid residues.
19. An isolated recombinant protein or combination for use according to any one of items 1-18 wherein the isolated recombinant protein is consisting of more than or equal to 90 amino acid residues.
20. An isolated recombinant protein or combination for use according to any one of the preceding items wherein said microbubbles and/or said nanodroplets are lipid coated.
21. An isolated recombinant protein or combination for use according to any one of the preceding items, wherein said microbubbles and/or nanodroplets comprise 1,2-distearyol-sn-glycero-3-phosphocoline (DSPC), 1,2-distearyol-sn-glycero-3-phosphoethanolamine-N-(metoxy (polyethyleneglycol) 2000) and a gas core of perfluorobutane.
22. An isolated recombinant protein or combination for use according to any one of the items 1-21, wherein said microbubbles and/or nanodroplets comprise sulphur hexafluoride, polyethylene glycol (PEG, Macrogol), distearylphosphatidylcholine (DSPC), sodium 1,2-dipalmitoyl-sn-glycero-3-phosphatidylglycerol and palmitic acid.
23. An isolated recombinant protein or combination for use according to any one of the preceding items wherein said microbubbles and/or said nanodroplets have a diameter in the range of 1-8 μm, such as 2-6 μm, such as 4-5 μm.
24. An isolated recombinant protein or combination for use according to any one of items 1-23, wherein said treatment is selected from the group consisting of preventive, palliative and curative treatment.
25. A method of treating Alzheimer's disease in a mammal, including man, in need thereof comprising the steps of;
   administrating to said mammal a plurality of lipid microbubbles and/or nanodroplets
   administrating to said mammal an isolated recombinant protein selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4);
wherein said isolated recombinant protein is not comprised within said microbubbles and/or said nanodroplets; and
wherein the method is not comprising any step of ultrasound treatment of any tissue of the mammal.
26. A method of treating Alzheimer's disease in a mammal, including man, in need thereof consisting of the steps of;
   administrating to said mammal a plurality of lipid microbubbles and/or nanodroplets
   administrating to said mammal an isolated recombinant protein selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4);
wherein said isolated recombinant protein is not comprised within said microbubbles and/or said nanodroplets.
27. A method according to any one items 25-26, wherein the isolated recombinant protein is selected from the group of proteins comprising an amino acid sequence having at least 70% identity to the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
28. A method according to item 27, wherein the isolated recombinant protein is selected from the group of proteins comprising an amino acid sequence having at least 80%, preferably at least 85%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
29. A method according to item 28, wherein the isolated recombinant protein is selected from the group of proteins comprising an amino acid sequence having at least 90%, preferably at least 95%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
30. A method according to item 29, wherein the isolated recombinant protein is selected from the group of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
31. A method according to any one of the items 25-26, wherein the isolated recombinant protein is selected from the group of proteins comprising an amino acid sequence having at least 70% identity to any one of residues 113-231 of Bri2 from human (SEQ ID NO: 2) and the BRICHOS domain of Bri2 from human (SEQ ID NO: 5).
32. A method according to item 31, wherein the isolated recombinant protein is selected from the group of proteins comprising an amino acid sequence having at least 80%, preferably at least 85%, identity to any one of residues 113-231 of Bri2 from human (SEQ ID NO: 2) and the BRICHOS domain of Bri2 from human (SEQ ID NO: 5).
33. A method according to item 31, wherein the isolated recombinant protein is selected from the group of proteins comprising an amino acid sequence having at least 90%, preferably at least 95%, preferably at least 99% identity to any one of residues 113-231 of Bri2 from human (SEQ ID NO: 2) and the BRICHOS domain of Bri2 from human (SEQ ID NO: 5).
34. A method according to item 33, wherein the isolated recombinant protein is selected from the group consisting of residues 113-231 of Bri2 from human (SEQ ID NO: 2); and the BRICHOS domain of Bri2 from human (SEQ ID NO: 5).
35. A method according to any one of items 25-34, wherein the amino acid residue corresponding to position 221 in SEQ ID NO: 1 is selected from the group consisting of Glu and Asp.
36. A method according to item 35, wherein the amino acid residue corresponding to position 221 in SEQ ID NO: 1 is Glu.
37. A method according to any one of the items 25-34, wherein the lipid microbubbles and/or nanodroplets are as defined in any one of items 20-23.
38. An isolated protein comprising
   (i) a first protein moiety selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); and
   (ii) a second protein or polypeptide moiety, preferably containing at least 50 amino acid residues;
wherein said isolated protein is not comprising an amino acid sequence having at least 70% identity to residues 1-89 of Bri2 from human (SEQ ID NO: 3); and
wherein said isolated protein is not comprising an amino acid sequence having at least 70% identity to human ABri23 (SEQ ID NO: 4).
39. An isolated protein according to item 38, wherein the first protein moiety is selected from the group of proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
40. An isolated protein according to item 39, wherein the first protein moiety is selected from the group of proteins comprising an amino acid sequence having at least 80%, preferably at least 85%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
41. An isolated protein according to item 40, wherein the first protein moiety is selected from the group of proteins comprising an amino acid sequence having at least 90% identity, preferably at least 95%, to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
42. An isolated protein according to item 41, wherein the first protein moiety is selected from the group of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
43. An isolated protein according to item 38, wherein the first protein moiety is selected from the group of proteins comprising an amino acid sequence having at least 70% identity to any one of residues 113-231 of Bri2 from human (SEQ ID NO: 2) and the BRICHOS domain of Bri2 from human (SEQ ID NO: 5).
44. An isolated protein according to item 43, wherein the first protein moiety is selected from the group of proteins comprising an amino acid sequence having at least 80%, preferably at least 85%, identity to any one of residues 113-231 of Bri2 from human (SEQ ID NO: 2) and the BRICHOS domains of Bri2 from human (SEQ ID NO: 5).
45. An isolated protein according to item 44, wherein the first protein moiety is selected from the group of proteins comprising an amino acid sequence having at least 90%, preferably at least 95%, identity to any one of residues 113-231 of Bri2 from human (SEQ ID NO: 2) and the BRICHOS domain of Bri2 from human (SEQ ID NO: 5).
46. An isolated protein according to item 45, wherein the first protein moiety is selected from the group consisting of residues 113-231 of Bri2 from human (SEQ ID NO: 2); and the BRICHOS domain of Bri2 from human (SEQ ID NO: 5).
47. An isolated protein according to any one of items 38-46, wherein the amino acid residue in the first protein moiety corresponding to position 221 in SEQ ID NO: 1 is selected from the group consisting of Glu and Asp.
48. An isolated protein according to item 47, wherein the amino acid residue in the first protein moiety corresponding to position 221 in SEQ ID NO: 1 is Glu.
49. An isolated protein according to any one of items 38-48, wherein the first protein moiety is consisting of less than or equal to 200 amino acid residues, such as less than or equal to 150 amino acid residues.
50. An isolated protein according to any one of items 38-48, wherein the first protein moiety is consisting of more than or equal to 90 amino acid residues.
51. An isolated protein according to any one of items 38-50, wherein said second protein or polypeptide moiety contains from 50 to 2000 amino acid residues, such as from 50 to 1000 amino acid residues, such as from 50 to 500 amino acid residues, such as from 50 to 100 amino acid residues.
52. An isolated protein according to any one of items 38-51, wherein the size of said second protein or polypeptide moiety is 5-200 kDa, such as 5-100 kDa, such as 5-50 kDa, such as 5-10 kDa.
53. An isolated protein according to any one of items 38-52, wherein the first protein moiety is linked directly or indirectly to the amino-terminal or the carboxy-terminal end of the second protein or polypeptide moiety.
54. An isolated protein according to any one of items 38-53, wherein the second protein or polypeptide moiety constitutes the amino-terminal and/or the carboxy-terminal end of the protein.
55. An isolated protein according to any one of items 38-54, wherein the second protein or polypeptide moiety is selected from the group consisting of protein drugs, polypeptide drugs, protein tags, fluorescent proteins, antibodies, enzymes and/or neurotrophic factors.
56. An isolated protein according to item 55, wherein the second protein or polypeptide moiety is an antibody.
57. An isolated protein according to item 55, wherein the second protein or polypeptide moiety is a neurotrophin selected from the group consisting of brain-derived neurotrophic factor (BDNF), nerve growth factor (NGF), neurotrophin-3, neurotrophin-4, ciliary neurotrophic factor (CNTF), glial cell line-derived neurotrophic factor (GDNF), ephrins, epidermal growth factor (EGF), transforming growth factor (TGF), insulin-like growth factor (IGF), vascular endothelial growth factor (VEGF), fibroblast growth factor (FGF), platelet-derived growth factor (PDGF), and/or interleukins.
58. An isolated protein according to item 55, wherein the second protein or polypeptide moiety is selected from the group consisting of α-L-iduronidase, Iduronate sulfatase, N-acetylgalactosamine 6-sulfatase, N-acetylgalactosamine 4-sulfatase, α-galactosidase, α-glucosidase, β-glucocerebrosidase and/or Lysosomal acid lipase.
59. An isolated protein according to any one of the items 38-58, wherein said isolated protein is a recombinant fusion protein.
60. An isolated protein according to any one of the items 38-58, wherein said first protein moiety is chemically linked to said second protein or polypeptide moiety.
61. A combination of an isolated protein according to any one of the items 38-60 and a plurality of lipid microbubbles and/or nanodroplets, wherein said isolated protein is not comprised within said microbubbles and/or said nanodroplets.
62. A kit comprising an isolated protein according to any one of the items 38-60 and a plurality of lipid microbubbles and/or nanodroplets, wherein said isolated protein is not comprised within said microbubbles and/or said nanodroplets.
63. A combination or a kit according to any one of the items 61-62, wherein the lipid microbubbles and/or nanodroplets are as defined in any one of items 20-23.
64. A method for transporting an isolated protein according to any one of items 38-60 across the blood-brain barrier in a mammal, including man, in need thereof consisting of the steps of;
   administrating to said mammal a plurality of lipid microbubbles and/or nanodroplets;
   administrating to said mammal said isolated protein.
65. A method according to item 64, wherein the method is not comprising any step of ultrasound treatment of any tissue of the mammal.
66. A method according to any one of the items 64-65, wherein the lipid microbubbles and/or nanodroplets are as defined in any one of items 20-23.
67. An isolated protein according to any one of the items 38-60 for use in a method of treatment involving transporting said isolated protein across the blood-brain barrier in a mammal, including man, in need thereof comprising the steps of:
   administrating to said mammal a plurality of lipid microbubbles and/or nanodroplets
   administrating to said mammal said isolated protein;
wherein said isolated protein is not comprised within said microbubbles and/or said nanodroplets.
68. An isolated protein for use in the method according to item 67, wherein the method is not comprising any step of ultrasound treatment of any tissue of the mammal.
69. An isolated protein according to any one of the items 38-60 for use in a method of treatment involving transporting said isolated protein across the blood-brain barrier in a mammal, including man, in need thereof consisting of the steps of:
   administrating to said mammal a plurality of lipid microbubbles and/or nanodroplets
   administrating to said mammal said isolated protein;
wherein said isolated protein is not comprised within said microbubbles and/or said nanodroplets.
70. An isolated protein for use according to any one of the items 67-69, wherein the lipid microbubbles and/or nanodroplets are as defined in any one of items 20-23.
71. A method for transporting an isolated protein according to any one of items 38-60 across the blood-brain barrier in a mammal, including man, in need thereof comprising the step of:
   administrating to said mammal said isolated protein.
72. A method for transporting an isolated protein according to any one of items 38-60 across the blood-brain barrier in a mammal, including man, in need thereof consisting of the step of:
   administrating to said mammal said isolated protein.
73. A method of treating a medical condition in a mammal, including man, in need thereof comprising the step of:
   administrating to said mammal an isolated protein according to any one of items 55-60.
74. A method of treating a medical condition in a mammal, including man, in need thereof consisting of the step:
   administrating to said mammal an isolated protein according to any one of items 55-60.
75. An isolated protein according to any one of items 55-60 for use as a medicament.
76. An isolated protein according to any one of items 55-60 for use in a method of treating a medical condition in a mammal, including man, in need thereof comprising the step of:
   administrating to said mammal an isolated protein according to any one of items 55-60.
77. An isolated protein according to any one of items 55-60 for use in a method of treating a medical condition in a mammal, including man, in need thereof consisting of the step of:
   administrating to said mammal an isolated protein according to any one of items 55-60.

1. An isolated protein comprising:
(i) a first protein moiety selected from a group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); and
(ii) a second protein or polypeptide moiety, preferably containing at least 50 amino acid residues, wherein the second protein or polypeptide moiety is a monoclonal antibody;
wherein said isolated protein is not comprising an amino acid sequence having at least 70% identity to residues 1-89 of Bri2 from human (SEQ ID NO: 3); and
wherein said isolated protein is not comprising an amino acid sequence having at least 70% identity to human ABri23 (SEQ ID NO: 4).

2. (canceled)

3. (canceled)

4. (canceled)

5. The isolated protein according to claim 1, wherein the monoclonal antibody is selected from aducanumab, gantenerumab, 3D6 (bapineuzumab), m266 (solanezumab), donanemab and lecanemab.

6. The isolated protein according to claim 5, wherein the monoclonal antibody is selected from aducanumab, gantenerumab, 3D6 (bapineuzumab), and m266 (solanezumab).

7. The isolated protein according to claim 5, wherein the antibody is donanemab.

8. The isolated protein according to claim 5, wherein the antibody is lecanemab.

9. (canceled)

10. (canceled)

11. (canceled)

12. (canceled)

13. The isolated protein according to claim 1, wherein said isolated protein is a recombinant fusion protein.

14. The isolated protein according to claim 1, wherein said first protein moiety is chemically linked to said second protein or polypeptide moiety.

15. (canceled)

16. The isolated protein according to claim 1, wherein the first protein moiety is selected from a group of proteins comprising an amino acid sequence having at least 80% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

17. (canceled)

18. (canceled)

19. (canceled)

20. The isolated protein according to claim 1, wherein the first protein moiety is selected from the group of proteins comprising an amino acid sequence having at least 80% identity to any one of residues 113-231 of Bri2 from human (SEQ ID NO: 2) and the BRICHOS domains of Bri2 from human (SEQ ID NO: 5).

21. The isolated protein according to claim 20, wherein the first protein moiety is selected from a group of proteins comprising an amino acid sequence having at least 90% identity to any one of residues 113-231 of Bri2 from human (SEQ ID NO: 2) and the BRICHOS domain of Bri2 from human (SEQ ID NO: 5).

22. The isolated protein according to claim 21, wherein the first protein moiety is selected from a group consisting of residues 113-231 of Bri2 from human (SEQ ID NO: 2); and the BRICHOS domain of Bri2 from human (SEQ ID NO: 5).

23. The isolated protein according to claim 1, wherein the amino acid residue in the first protein moiety corresponding to position 221 in SEQ ID NO: 1 is selected from a group consisting of Glu and Asp.

24. The isolated protein according to claim 23, wherein the amino acid residue in the first protein moiety corresponding to position 221 in SEQ ID NO: 1 is Glu.

25. The isolated protein according to claim 1, wherein the first protein moiety is consisting of less than or equal to 200 amino acid residues, such as less than or equal to 150 amino acid residues.

26. The isolated protein according to claim 1, wherein the first protein moiety is consisting of more than or equal to 90 amino acid residues.

27. (canceled)

28. (canceled)

29. The isolated protein according to claim 1, wherein the first protein moiety is linked directly or indirectly to the amino-terminal or the carboxy-terminal end of the second protein or polypeptide moiety.

30. The isolated protein according to claim 1, wherein the second protein or polypeptide moiety constitutes the amino-terminal and/or the carboxy-terminal end of the protein.

31-44. (canceled)

45. A method for transporting an isolated protein according to claim 1 across the blood-brain barrier in a mammal, including man, in need thereof comprising the step of:
administrating to said mammal said isolated protein.

46. A method of treating a medical condition in a mammal, including man, in need thereof comprising the step of:
administrating to said mammal an isolated protein according to claim 1.

47. The method according to claim 46, wherein the medical condition is Alzheimer's disease.
